(19) United States
(12) Patent Application Publication
(10) Pub. No.: US 2008/0305127 A1
(43) Pub. Date: Dec. 11, 2008

(54) Conjugate Vaccines

(75) Inventor: Jan Poolman, Rixensart (BE)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(73) Assignee: Glaxosmithkline Biologicals S.A., Rixensart (BE)

(21) Appl. No.: 12/096,852

(22) PCT Filed: Dec. 22, 2006
(86) PCT No.: PCT/EP06/70173

§ 371 Date: Jun. 10, 2008

(30) Foreign Application Priority Data
Dec. 23, 2005 (GB) 0526412.2
Apr. 7, 2006 (GB) 0607088.2

(51) Int. Cl. A61K 39/095; A61K 39/02

(52) U.S. Cl. 424/194.1; 424/197.11

(57) ABSTRACT

The invention provides vaccines against Neisseria meningitidis, pneumococcus and DTPa/w. In particular, it provides vaccines based on conjugated capsular saccharides from multiple meningococcal and/or pneumococcal serogroups. It further provides vaccine administration schemes for the immunisation of human patients with two or more of these vaccines.

### TECHNICAL FIELD

This invention concerns vaccines against Neisseria meningitis and pneumococcus. In particular, it concerns vaccines based on conjugated capsular saccharides from multiple meningococcal and/or pneumococcal serogroups.

### BACKGROUND ART

Based on the organism's capsular polysaccharide, twelve serogroups of N. meningitidis have been identified (A, B, C, H, I, K, L, 29E, W135, X, Y and Z). Group A is the pathogen most often implicated in epidemic disease in sub-Saharan Africa. Serogroups B and C are responsible for the vast majority of cases in USA and in most developed countries. Serogroups W135 and Y are responsible for the remaining cases in USA and developed countries.

A tetravalent vaccine of capsular polysaccharides from serogroups A, C, Y and W135 has been known for many years [1,2]. Although effective in adolescents and adults' it induces a poor immune response and short duration of protection and cannot be used in infants [e.g ref. 3] because polysaccharides are T cell-independent antigens that induce a weak immune response which cannot be boosted. The polysaccharides in this vaccine are not conjugated [4].

Conjugate vaccines against serogroup C have been approved for human use, and include Menjugate™ [5], Meningitec™ and NeisVac-C™. Mixtures of conjugates from serogroups A+C are known [6-8] and mixtures of conjugates from serogroups A+C+W135+Y have been reported [9-13].

While meningococcal conjugates are well known, they have not yet been fitted into existing pediatric immunisation schedules, which for developed countries typically involve: hepatitis B vaccine at birth; and, starting at 2 months, all of diphtheria/tetanus/pertussis (D-T-P), H. influenzae type b (Hib) conjugate, inactivated poliovirus and pneumococcus conjugates at 2 months.

When adding conjugated vaccines to existing immunisation schedules, however, the issue of carrier-induced epitopic suppression (or “carrier suppression”, as it is generally known) must be addressed, particularly suppression arising from carrier priming. “Carrier suppression” is the phenomenon whereby pre-immunisation of an animal with a carrier protein prevents it from later eliciting an immune response against a new antigenic epitope that is presented on that carrier [14].

As reported in reference 15, where several vaccine antigens contain the same protein component (being used as an immunogen and/or as a carrier protein in a conjugate) then there is the potential for interference between those antigens. In reference 15, the immune response against an antigen that was conjugated to a tetanus toxoid (Tt) carrier was suppressed by pre-existing immunity against Tt.

Reference 16 reports how a combination of D-T-P vaccines with a Hib conjugate vaccine was adversely affected where the carrier for the Hib conjugate was the same as the tetanus antigen from the D-T-P vaccine. The authors concludes that this “carrier suppression” phenomenon, arising from interference by a common protein carrier, should be taken into account when introducing vaccines that include multiple conjugates.

In contrast to references 15 and 16, reference 17 reported that priming with tetanus toxoid had no negative impact on the immune response against a subsequently-administered Hib-Tt conjugate, but suppression was seen in patients with maternally acquired anti-Tt antibodies. In reference 18, however, an “epitopic suppression” effect was reported for a Tt-based peptide conjugate in patients having existing anti-Tt antibodies resulting from tetanus vaccination.

In reference 19, it was suggested that a conjugate having CRM197 (a detoxified mutant of diphtheria toxin) as the carrier may be ineffective in children that had not previously received diphtheria toxin as part of a vaccine (e.g as part of a D-T-P or D-T vaccine). This work was further developed in reference 20, where a carrier priming effect by D-T immunization was seen to persist for subsequent immunization with Hib conjugates. In reference 21, the authors found that pre-immunisation with a diphtheria or tetanus toxoid carrier protein reduced the increase in anti-Hib antibody levels after a subsequent immunization with the Hib capsular saccharide conjugated to those carriers, with IgG1 and IgG2 being equally affected.

Responses to the carrier portions of the conjugates were also suppressed. Furthermore, a more general non-epitope-specific suppression was seen, as pre-immunisation with one conjugate was seen to affect immune responses against both the carrier and saccharide portions of a second conjugate that was administered four weeks later.

The use of different carrier proteins in a single multivalent pneumococcal conjugate vaccine is reported in reference 22, with multiple carriers being used in order to avoid carrier suppression. The authors predict that there is a maximum load of a carrier protein that can be tolerated in a multivalent conjugate vaccine without giving rise to negative interference. In reference 23 it was reported that pneumococcal conjugate vaccines including mixed carrier proteins elicited, in parallel to the anti pneumococcus response, unintentional booster responses to the carriers.

In reference 24, an investigation of whether diphtheria and tetanus boosters could be administered with monovalent meningococcal serogroup C conjugates, it was found that titres against the meningococcal conjugate were reduced where the carrier was tetanus toxoid carrier and the patient had received prior immunization with a tetanus-containing vaccine.

Finally, reference 25 reports that “prior exposure to the carrier protein can either enhance or suppress antibody response to polysaccharides administered in saccharide-protein conjugates”. The conjugates used in reference 25 used tetanus toxoid or the CRM197 mutant as carrier protein.

The situation concerning carrier priming and/or suppression is thus confused, and it remains unclear whether any particular conjugate will suffer from carrier suppression or will benefit from a carrier priming enhancement. Meningococcal conjugate vaccines will not be in a position to be integrated into or added to existing pediatric immunization schedules until this issue is addressed. Furthermore, as some meningococcal conjugates are to be administered as tetravalent mixtures (i.e. four different conjugates) then the potential for carrier suppression becomes even more of a risk.

In addition to the problem of priming with a carrier having a negative impact on immune responses against saccharide conjugates, the reverse can also occur i.e. immunization with a conjugate can have a negative impact on immune responses against the carrier [26].

### DISCLOSURE OF THE INVENTION

### First Aspect of the Invention

It has now been found that meningococcal conjugates (on a tetanus toxoid carrier) can be administered to patients even where they have already received the carrier protein, either in the form of a previous immunogen (e.g in a D-T-P or a D-T immunization) or as a previous carrier protein (e.g in a Hib conjugate or pneumococcal conjugate vaccine).

The invention thus provides a method for immunizing a human patient against a disease caused by Neisseria meningitidis, comprising the step of administering to the human patient a composition that comprises at least two of: (a) a conjugate of (i) the capsular saccharide of serogroup A N.meningitidis and (ii) a tetanus toxoid or derivative thereof; (b) a conjugate of (i) the capsular saccharide of serogroup C N.meningitidis and (ii) a tetanus toxoid or derivative thereof; (c) a conjugate of (i) the capsular saccharide of serogroup W135 N.meningitidis and (ii) a tetanus toxoid or derivative thereof; and (d) a conjugate of (i) the capsular saccharide of serogroup Y N.meningitidis and (ii) a tetanus toxoid or derivative thereof, wherein the patient has been pre-immunised with (a) a tetanus toxoid or derivative thereof and/or (b) a conjugate of (i) a capsular saccharide of an organism other than N.meningitidis and (ii) a tetanus toxoid or derivative thereof.

It also provides a method for immunizing a human patient against a disease caused by Neisseria meningitidis, comprising the step of administering to the human patient a composition that comprises at least two of: (a) a conjugate of (i) the capsular saccharide of serogroup A N.meningitidis and (ii) a tetanus toxoid; (b) a conjugate of (i) the capsular saccharide of serogroup C N.meningitidis and (ii) a tetanus toxoid; (c) a conjugate of (i) the capsular saccharide of serogroup W135 N.meningitidis and (ii) a tetanus toxoid; and (d) a conjugate of (i) the capsular saccharide of serogroup Y N.meningitidis and (ii) a tetanus toxoid, wherein the patient has been pre-immunised with (a) a tetanus toxoid and/or (b) a conjugate of (i) a capsular saccharide of an organism other than N. meningitidis and (ii) a tetanus toxoid.

Furthermore there is provided a use of at least two of: (a) a conjugate of (i) the capsular saccharide of serogroup A N.meningitidis and (ii) a tetanus toxoid or derivative thereof; (b) a conjugate of (i) the capsular saccharide of serogroup C N.meningitidis and (ii) a tetanus toxoid or derivative thereof; (c) a conjugate of (i) the capsular saccharide of serogroup W135 N. meningitidis and (ii) a tetanus toxoid or derivative thereof; and (d) a conjugate of (i) the capsular saccharide of serogroup Y N.meningitidis and (ii) a tetanus toxoid or derivative thereof, in the manufacture of a medicament for immunizing a human patient against a disease caused by Neisseria meningitidis, wherein the patient has been pre-immunised with (a) a tetanus toxoid or derivative thereof and/or (b) a conjugate of (i) a capsular saccharide of an organism other than N.meningitidis and (ii) a tetanus toxoid or derivative thereof.

There is also provided a use of at least two of: (a) a conjugate of (i) the capsular saccharide of serogroup A N.meningitidis and (ii) a tetanus toxoid; (b) a conjugate of (i) the capsular saccharide of serogroup C N. meningitidis and (ii) a tetanus toxoid; (c) a conjugate of (i) the capsular saccharide of serogroup W135 N.meningitidis and (ii) a tetanus toxoid; and (d) a conjugate of (i) the capsular saccharide of serogroup Y N.meningitidis and (ii) a tetanus toxoid, in the manufacture of a medicament for immunizing a human patient against a disease caused by Neisseria meningitidis, wherein the patient has been pre-immunised with (a) a tetanus toxoid and/or (b) a conjugate of (i) a capsular saccharide of an organism other than N.meningitidis and (ii) a tetanus toxoid.

The meningococcal disease is preferably meningitis, more preferably bacterial meningitis, and most preferably meningococcal meningitis. Thus the invention can be used to protect against meningococcal infections that cause meningitis.

Where the pre-immunisation antigen is a derivative of a tetanus toxoid (TT) then that derivative preferably remains immunologically cross-reactive with TT, and is preferably fragment C.

### The Pre-Immunised Patient

The patient to be immunised has been pre-immunised with: (a) a tetanus toxoid or derivative thereof, and/or (b) a conjugate of (i) a capsular saccharide of an organism other than Neisseria meningitidis and (ii) a tetanus toxoid or derivative thereof. Typical pre-immunisation will have included: a tetanus toxoid antigen, a Hib capsular saccharide conjugate using a tetanus toxoid carrier, and/or a pneumococcal capsular saccharide conjugate using a tetanus toxoid carrier.

The patient will have received at least one (e.g 1, 2, 3 or more) dose of the pre-immunisation antigen(s), and that dose (or the earliest of multiple doses) will have been administered to the patient at least 0.5, 1, 2, 4 or at least six (e.g 6, 9, 12, 15, 18, 21, 24, 36, 48, 60, 120, 180, 240, 300 or more) months before the immunization with the meningococcal conjugates according to the invention. In a preferred group of patients, the pre-immunisation took place within 3 years of birth e.g within 2 years of birth, within 1 year of birth, within 6 months of birth, or even within 3 months, 2 months or 1 month of birth.

The patient to be immunised according to the invention will typically be a human. The human will generally be at least 1 month old e.g at least 2 months old, at least 3 months old, at least 4 months old, at least 6 months old, at least 2 years old, at least 5 years old, at least 11 years old, at least 17 years old, at least 40 years old, at least 55 years old, etc. A preferred set of patients is at least 6 months old. Another preferred set of patients is in the age group 2-55 years old, and another preferred set of patients is in the age group 11-55 years old. A further preferred set of patients is less than 11 years old e.g 2-11 years old. In all cases, however, regardless of age, the patient will have been pre-immunised as defined herein.

Where the pre-immunisation antigen is a tetanus toxoid then the patient will typically have received the toxoid as the ‘T’ antigen in a D-T-P or a D-T pre-immunisation. Such immunizations are typically given to newborn children at ages 2, 3, and 4 months. Where the immunization includes a pertussis vaccine, that vaccine may be a whole cell or cellular pertussis vaccine (‘Pw’), but is preferably an acellular pertussis vaccine (‘Pa’). Pre-immunisation Pa vaccines will generally include one, two or three of the following well-known and well-characterised B.pertussis antigens: (1) pertussis toxoid (‘PT’), detoxified either by chemical means or by site-directed mutagenesis eg the 9K/129G′ mutant [30], (2) filamentous hemagglutinin (‘FHA’), (3) pertactin (also known as ‘69 kiloDalton outer membrane protein’). Acellular pertussis vaccines may also include agglutinogen 2 and/or agglutinogen 3. The ‘D’ antigen in a D-T-P pre-immunisation is typically a diphtheria toxoid.

Where the pre-immunisation antigen is a tetanus toxoid then the patient may also or alternatively have received the toxoid as the carrier protein of a protein-saccharide conjugate. Such conjugates include the ‘PRP-T’ Hib conjugate.

Where the pre-immunisation antigen is tetanus toxoid then the patient will typically have been pre-immunised with a Hib conjugate and/or a multivalent pneumococcal conjugate. Such immunizations are typically given to newborn children at ages 2, 3, and 4 months. Hib conjugates are well know (reference 32). Pneumococcal conjugates may also use a Tetanus toxoid carrier for one or more of the saccharides. The patient may also have been pre-immunised with a serogroup C meningococcal (‘MenC’) conjugate. MenC conjugates that use tetanus toxoid as a carrier. Preferably, however, the patient has been pre-immunised with Hib and/or pneumococcal conjugate, but not with a MenC conjugate. If the patient has been pre-immunised with a MenC conjugate then the vaccine administered according to the invention may or may not include a serogroup C conjugate.

Tetanus toxoid is a well known and well characterized protein that can be obtained by treating the toxin with an inactivating chemical, such as formalin or formaldehyde.

The result of the pre-immunisation is that the patient's immune system has been exposed to the pre-immunisation antigens. For pre-immunisation with tetanus toxoid (Tt), this generally means that the patient will have raised an anti-Tt antibody response (typically to give an anti-Tt titer>0.01 IU/ml) and will possess memory B and/or T lymphocytes specific for Tt i.e. pre-immunisation with Tt is typically adequate to elicit an anamnestic anti-Tt immune response in the patient. For pre-immunisation where Tt (or derivative) is a carrier for a saccharide within a conjugate then the pre-immunisation will have raised an anti-saccharide response and the patient will possess memory B and/or T lymphocytes specific for the saccharide i.e. the pre-immunisation is typically adequate to elicit an anamnestic anti-saccharide immune response in the patient. The pre-immunisation was preferably adequate to elicit protective immunity in the patient e.g. against tetanus disease.

Thus the patients to be immunised according to the invention are distinct from patients in general, as they are members of a subset of the general population whose immune systems have already mounted an immune response to the pre-immunisation antigens, such that immunization according to the invention with a meningococcal conjugate that includes a tetanus toxoid (or derivative thereof) carrier elicits a different immune response in the subset than in patients who have not previously mounted an immune response to the pre-immunisation antigens. Patients who have been pre-immunised with Tt (or derivative) as the carrier of a conjugate (particularly of a Hib conjugate) are preferred. Particularly preferred patients have been pre-immunised with Tt (or derivative) as the carrier of a conjugate and also with Tt as an unconjugated immunogen.

As well as having been pre-immunised with a tetanus toxoid (or derivative), in conjugated or non-conjugated form, the patient may have been pre-immunised with other antigens. Such antigens include, but are not limited to: pertussis antigen(s)—see above; diphtheria toxoid—see above; Haemophilus influenzae type B—see above; hepatitis B surface antigen (HBsAg); poliovirus, such as an inactivated poliovirus vaccine (IPV); Streptococcus pneumoniae—see above; influenza virus; BCG; hepatitis A virus antigens; measles virus; mumps virus; rubella virus; varicella virus; etc. The patient may or may not have been pre-immunised with one or more meningococcal capsular saccharide conjugate(s).

In some preferred embodiments, at the time when a patient first receives a meningococcal conjugate, they have already been pre-immunised with Tt (or derivative). In other embodiments, a meningococcal conjugate is administered to a patient who has already been pre-immunised with both (i) Tt or a derivative and (ii) a meningococcal conjugate.

### The Conjugates

The invention immunizes patients with conjugated saccharides. Conjugation is used to enhance the immunogenicity of saccharides, as it converts them from T-independent antigens to T-dependent antigens, thus allowing priming for immunological memory. Conjugation is particularly useful for pediatric vaccines [e.g. ref. 37] and is a well known technique [e.g reviewed in refs. 38 to 46].

The composition used according to the invention comprises at least two meningococcal conjugates, wherein each conjugate comprises a tetanus toxoid (or derivative thereof) carrier protein, and the capsular saccharide. The capsular saccharides are chosen from meningococcal serogroups A, C, W135 and Y, such that the compositions include saccharides from 2, 3, or all 4 of these four serogroups.

Specific compositions comprise saccharides from: serogroups A & C, serogroups A & W; serogroups A & Y; serogroups C & W135; serogroups C & Y. serogroups W135 & Y; serogroups A & C & W135; serogroups A & C & Y; serogroups A & W135 & Y; serogroups C & W135 & Y; serogroups A & C & W135 & Y. Compositions including saccharides from all four serogroups are most preferred.

The capsular saccharides of each of these four serogroups are well characterized. The capsular saccharide of serogroup A meningococcus is a homopolymer of (α1→6)-linked N-acetyl-D-mannosamine-1-phosphate, with partial O-acetylation in the C3 and C4 positions. The acetyl groups can be replaced with blocking groups to prevent hydrolysis [47], and such modified saccharides are still serogroup A saccharides within the meaning of the present invention. The serogroup C capsular saccharide is a homopolymer of (α2→9)-linked sialic acid (N-acetyl neuraminic acid, or ‘NeuNAc’). Most serogroup C strains have O-acetyl groups at C-7 and/or C-8 of the sialic acid residues, but about 15% of clinical isolates lack these O— acetyl groups [48,49]. The saccharide structure is written as →9)-Neu p NAc 7/8 OA(α2→. The serogroup W135 saccharide is a polymer of sialic acid galactose disaccharide units. Like the serogroup C saccharide, it has variable O-acetylation, but at sialic acid 7 and 9 positions [50]. The structure is written as: →4)-D-Neu p5Ac(7/9OAc)-α-(2→6) D-Gal-α-(1→. The serogroup Y saccharide is similar to the serogroup W135 saccharide, except that the disaccharide repeating unit includes glucose instead of galactose. Like serogroup W135, it has variable O-acetylation at sialic acid 7 and 9 positions [50]. The serogroup Y structure is written as: →4)-D-Neu p5Ac(7/90Ac)-α-(2→6) D-Glc-α-(1→.

The saccharides used according to the invention may be O-acetylated as described above (e.g with the same O-acetylation pattern as seen in native capsular saccharides), or they may be partially or totally de-O-acetylated at one or more positions of the saccharide rings, or they may be hyper-O-acetylated relative to the native capsular saccharides.

The saccharides used according to the invention may be shorter than the native capsular saccharides seen in bacteria. Thus the saccharides may be depolymerized, with depolymerization occurring after purification but before conjugation. Depolymerization reduces the chain length of the saccharides. One depolymerization method involves the use of hydrogen peroxide [9]. Hydrogen peroxide is added to a saccharide (e.g to give a final H2O2 concentration of 1%), and the mixture is then incubated (e.g at about 55° C.) until a desired chain length reduction has been achieved. Another depolymerization method involves acid hydrolysis [10]. Other depolymerization methods are known to the skilled person. The saccharides used to prepare conjugates for use according to the invention may be obtainable by any of these depolymerization methods. Depolymerization can be used in order to provide an optimum chain length for immunogenicity and/or to reduce chain length for physical manageability of the saccharides.

Typical carrier proteins for use in conjugates are bacterial toxins or toxoids, such as diphtheria toxin (or its CRM97 mutant) and tetanus toxin. Other known carrier proteins include the N.meningitidis outer membrane protein, synthetic peptides, heat shock proteins, pertussis proteins, cytokines, lymphokines, hormones, growth factors, artificial proteins comprising multiple human CD4+ T cell epitopes from various pathogen-derived antigens, protein D from non-typeable H.influenzae, pneumococcal surface protein PspA, iron-uptake proteins, toxin A or B from C.difficile, etc. According to the invention, however, the meningococcal conjugates include a tetanus toxoid (or derivative thereof, such as fragment C) carrier protein. Covalent conjugation is preferred.

It is possible to use more than one carrier protein in the compositions. Thus different carrier proteins can be used for different serogroups e.g serogroup A saccharides might be conjugated to tetanus toxoid while serogroup C saccharides might be conjugated to diphtheria toxoid. It is also possible to use more than one carrier protein for a particular saccharide antigen e.g serogroup A saccharides might be in two groups, with some conjugated to tetanus toxoid and others conjugated to diphtheria toxoid. In general, however, it is preferred to use the same carrier protein for all meningococcal saccharides in the composition, and more preferably for all saccharides (i.e. including any non-meningococcal conjugates that may be present). It is preferred that compositions/medicaments of this aspect of the invention do not include any diphtheria tetanus toxoid or CRM197 carrier protein.

A single carrier protein might carry more than one saccharide antigen [51]. For example, a single carrier protein might have conjugated to it saccharides from serogroups A and C. To achieve this goal, saccharides can be mixed prior to the conjugation reaction. In general, however, it is preferred to have separate conjugates for each serogroup. Conjugates are preferably mixed to give substantially a 1:1:1:1 ratio (measured as mass of saccharide) e.g the mass of each serogroup's saccharide is within +10% of each other. A typical quantity of meningococcal antigen per serogroup in a composition is between 1 μg and 20 μg e.g between 2 and 10 μg per serogroup, or about 4 or 5 μg. As an alternative to a 1:1:1:1 ratio, a double serogroup A dose may be used (2:1:1:1).

Conjugates with a saccharide:protein ratio (w/w) of between 1:15 (i.e. excess protein) and 15:1 (i.e. excess saccharide), preferably between 1:10 and 10:1, more preferably between 1:5 and 5:1, are preferred. Excess carrier protein may be used for instance 1:3.

Conjugates may be used in conjunction with free carrier protein [52]. When a given carrier protein is present in both free and conjugated form in a composition of the invention, however, the; unconjugated form is preferably no more than 5% of the total amount of the carrier protein in the composition as a whole, and more preferably present at less than 2% by weight. Similarly, unconjugated saccharide is preferably no more than 15% by weight of the total amount of saccharide.

Any suitable conjugation reaction can be used, with any suitable linker where necessary.

The saccharide will typically be activated or functionalised prior to conjugation. Activation may involve, for example, cyanylating reagents such as CDAP (e.g. 1-cyano-4-dimethylamino pyridinium tetrafluoroborate [53, 54, etc.]). Other suitable techniques use carbodiimides, hydrazides, active esters, norborane, p-nitrobenzoic acid, N-hydroxysuccinimide, S—NHS, EDC, TSTU; see also the introduction to reference 44).

Linkages via a linker group may be made using any known procedure, for example, the procedures described in references 55 and 56. One type of linkage involves reductive amination of the polysaccharide, coupling the resulting amino group with one end of an adipic acid linker group, and then coupling a protein to the other end of the adipic acid linker group [42, 57, 58]. Other linkers include B-propionamido [59], nitrophenyl-ethylamine [60], haloacyl halides [61], glycosidic linkages [62], 6-aminocaproic acid [63], ADH [64], C4 to C12 moieties [65] etc. As an alternative to using a linker, direct linkage can be used. Direct linkages to the protein may comprise oxidation of the polysaccharide followed by reductive amination with the protein, as described in, for example, references 66 and 67.

A process involving the introduction of amino groups into the saccharide (e.g. by replacing terminal ═O groups with —NH2) followed by derivatisation with an adipic diester (e.g adipic acid N— hydroxysuccinimide diester) and reaction with carrier protein may be done.

In one conjugation method, a saccharide is reacted with adipic acid dihydrazide. For serogroup A, carbodiimide may also be added at this stage. After a reaction period, sodium cyanoborohydride is added. Derivatised saccharide can then be prepared e.g by ultrafiltration. The derivatized saccharide is then mixed with carrier protein (e.g. with a tetanus toxoid), and carbodiimide is added. After a reaction period, the conjugate can be recovered. Further details of this conjugation method can be found in reference 10. Conjugates obtainable by this method may be used according to the invention e.g conjugates comprising a tetanus toxoid carrier and an adipic acid linker. Conjugates are preferably prepared separately and then mixed. After mixing, the concentration of the mixed conjugates can be adjusted e.g with sterile pyrogen-free, phosphate-buffered saline. Each conjugate, before mixing, preferably contains no more than 15 μg of carrier.

The result of administering meningococcal conjugates according to the invention is preferably that, for each administered serogroup, the patient raises a serum bactericidal antibody (SBA) response; with the increase in SBA titre (compared to the pre-immunised patient before receiving the mixed meningococcal conjugates) being at least 4-fold, and preferably at least 8-fold. The SBA test is a standard correlate for meningococcal protection. Further details of serologic correlates for meningococcal vaccines are given in reference 68.

### Further Antigenic Components of Compositions Used According to the Invention

In addition to meningococcal conjugates, compositions used according to the invention may optionally include 1, 2 or 3 of the following further antigens:

1. A conjugated capsular saccharide from S. pneumonia [e.g chapter 23 of ref. 32; refs. 69-71].

It is preferred to include saccharides from more than one serotype of S. pneumoniae. For example; mixtures of polysaccharides from 23 different serotype are known, as are conjugate vaccines with polysaccharides from between 5 and 11 different serotypes [72]. For example, PrevNar™ [31] contains antigens from seven serotypes (4, 6B, 9V, 14, 18C, 19F, and 23F) with each saccharide individually conjugated to CRM197 by reductive amination, with 2 μg of each saccharide per 0.5 ml dose (4 μg of serotype 6B), and with conjugates adsorbed on an aluminium phosphate adjuvant. Where pneumococcal conjugates are included in a compositions for use with the invention, the composition preferably includes at least serotypes 6B, 14, 19F and 23F. One or more serotypes may be conjugated to tetanus toxoid.

2. A conjugated capsular saccharide from H.influenzae B [e.g chapter 14 of ref. 32].

The carrier protein for the conjugate may be CRM197, Dt, a tetanus toxoid or an outer membrane complex of N.meningitidis. The saccharide moiety of the conjugate may be a polysaccharide (e.g full-length polyribosylribitol phosphate (PRP)), but can also have undergone depolymerization of the capsular polysaccharides to form oligosaccharides (e.g. MW from 1 to 5 kDa). One Hib conjugate comprises an oligosaccharide covalently linked to CRM197 via an adipic acid linker [73,74]. Another uses Tetanus toxoid instead. Administration of the Hib antigen preferably results in an anti-PRP antibody concentration of >0.15 μg/ml, and more preferably >1 μg/ml. Where a composition includes a Hib saccharide antigen, it preferably does not also include an aluminium hydroxide adjuvant. If the composition includes an aluminium phosphate adjuvant then the Hib antigen may be adsorbed to the adjuvant [75] or it may be non-adsorbed [27]. Prevention of adsorption can be achieved by selecting the correct pH during antigen/adjuvant mixing, an adjuvant with an appropriate point of zero charge, and an appropriate order of mixing for the various different antigens in a composition [76].

3. A Protein Antigen from Neisseria Meningitidis Serogroup B [e.g ref. 77].

The composition may comprise one or more of these further antigens. It may be an outer membrane vesicle preparation.

Such antigens may or may not be adsorbed to an aluminium salt.

If meningococcal conjugates are being administered in a series of doses then none, some or all of the doses may include these extra antigens.

Compositions containing the meningococcal conjugates preferably do not include diphtheria toxoid nor CRM197.

They preferably do not include pertussis antigens. They preferably do not include hepatitis B virus surface antigen. They preferably do not include poliovirus. A composition preferably contains no more than 50 μg of tetanus toxoid per meningococcal conjugate, and more preferably no more than 50 μg of tetanus toxoid for all meningococcal conjugates combined.

### Second Aspect of the Invention

As reported above, pneumococcal vaccination now tends to occur concomitantly with the first primary immunisation vaccination (around 2 months of age). The inventors believe that pre-immunisation with one or more carriers used for the pneumococcal conjugate vaccine may be useful.

This in a further aspect of the invention there is provided a method for immunizing a human patient against a disease caused by Streptococcus pneumoniae, comprising the step of administering to the human patient a composition that comprises at least seven, ten, eleven, thirteen or fourteen conjugates of different capsular saccharide serotypes of pneumococcus, at least one of which conjugated to a diphtheria toxoid or CRM197 or a derivative thereof, wherein the patient has been pre-immunised with (a) a diphtheria toxoid or derivative thereof and/or (b) a conjugate of (i) a capsular saccharide of an organism other than pneumococcus and (ii) a diphtheria toxoid or CRM197 or derivative thereof.

There is also provided a method for immunizing a human patient against a disease caused by Streptococcus pneumoniae, comprising the step of administering to the human patient a composition that comprises at least seven, ten, eleven, thirteen or fourteen conjugates of different capsular saccharide serotypes of pneumococcus, at least one of which conjugated to tetanus toxoid or a derivative thereof, wherein the patient has been pre-immunised with (a) a tetanus toxoid or derivative thereof and/or (b) a conjugate of (i) a capsular saccharide of an organism other than pneumococcus and (ii) a tetanus toxoid or derivative thereof.

In one embodiment of the invention there is provided a method for immunizing a human patient against a disease caused by Streptococcus pneumoniae, comprising the step of administering to the human patient a composition that comprises at least seven, ten, eleven, thirteen or fourteen conjugates of different capsular saccharide serotypes of pneumococcus, at least one of which is conjugated to tetanus toxoid or a derivative thereof and at least one of which is conjugated to diphtheria toxoid or CRM197 or a derivative thereof, wherein the patient has been pre-immunised with (a) a tetanus toxoid or derivative thereof and/or (b) a conjugate of (i) a capsular saccharide of an organism other than pneumococcus and (ii) a tetanus toxoid or derivative thereof and (c) a diphtheria toxoid or derivative thereof and/or (d) a conjugate of (i) a capsular saccharide of an organism other than pneumococcus and (ii) a diphtheria toxoid or CRM197 or derivative thereof.

Corresponding uses are also provided: the use of at least seven, ten, eleven, thirteen or fourteen conjugates of different capsular saccharide serotypes of pneumococcus, at least one of which conjugated to a diphtheria toxoid or CRM197 or a derivative thereof, in the manufacture of a medicament for immunizing a human patient against a disease caused by pneumococcus, wherein the patient has been pre-immunised with (a) a diphtheria toxoid or derivative thereof and/or (b) a conjugate of (i) a capsular saccharide of an organism other than pneumococcus and (ii) a diphtheria toxoid or CRM197 or derivative thereof; the use of at least seven, ten, eleven, thirteen or fourteen conjugates of different capsular saccharide serotypes of pneumococcus, at least one of which conjugated to a tetanus toxoid or a derivative thereof, in the manufacture of a medicament for immunizing a human patient against a disease caused by pneumococcus, wherein the patient has been pre-immunised with (a) a tetanus toxoid or derivative thereof and/or (b) a conjugate of (i) a capsular saccharide of an organism other than pneumococcus and (ii) a tetanus toxoid or derivative thereof; and the use of at least seven, ten, eleven, thirteen or fourteen conjugates of different capsular saccharide serotypes of pneumococcus, at least one of which is conjugated to tetanus toxoid or a derivative thereof and at least one of which is conjugated to diphtheria toxoid or CRM197 or a derivative thereof, in the manufacture of a medicament for immunizing a human patient against a disease caused by pneumococcus, wherein the patient has been pre-immunised with (a) a tetanus toxoid or derivative thereof and/or (b) a conjugate of (i) a capsular saccharide of an organism other than pneumococcus and (ii) a tetanus toxoid or derivative thereof and (c) a diphtheria toxoid or derivative thereof and/or (d) a conjugate of (i) a capsular saccharide of an organism other than pneumococcus and (ii) a diphtheria toxoid or CRM197 or derivative thereof.

Where the pre-immunisation antigen is a derivative of a diphtheria toxoid then that derivative preferably remains immunologically cross-reactive with Dt, and is preferably CRM197. Where the pre-immunisation antigen is a derivative of a tetanus toxoid then that derivative preferably remains immunologically cross-reactive with Tt, and is preferably fragment C.

### The Pre-Immunised Patient

The patient to be immunised has been pre-immunised with: (a) a diphtheria toxoid or derivative thereof, and/or (b) a conjugate of (i) a capsular saccharide of an organism other than pneumococcus and (ii) a diphtheria toxoid or derivative thereof, and/or c) a tetanus toxoid or derivative thereof, and/or (d) a conjugate of (i) a capsular saccharide of an organism other than pneumococcus and (ii) a tetanus toxoid or derivative thereof. Typical pre-immunisation will have included: a diphtheria toxoid antigen, a tetanus toxoid antigen, a Hib capsular saccharide conjugate using a diphtheria toxoid or CRM197 carrier or tetanus toxoid carrier, and/or a meningococcal capsular saccharide conjugate using a diphtheria toxoid or CRM197 carrier or tetanus toxoid.

The patient will have received at least one (e.g 1, 2, 3 or more) dose of the pre-immunisation antigen(s), and that dose (or the earliest of multiple doses) will have been administered to the patient at least 0.5, 1, 2, 4 or at least six (e.g 6, 9, 12, 15, 18, 21, 24, 36, 48, 60, 120, 180, 240, 300 or more) months before the immunization with the meningococcal conjugates according to the invention. In a preferred group of patients, the pre-immunisation took place within 3 years of birth e.g within 2 years of birth, within 1 year of birth, within 6 months of birth, or even within 3 months, 2 months or 1 month of birth.

The patient to be immunised according to the invention will typically be a human. The human will generally be at least 1 month old e.g at least 2 months old, at least 3 months old, at least 4 months old, at least 6 months old, at least 2 years old, at least 5 years old, at least 11 years old, at least 17 years old, at least 40 years old, at least 55 years old, etc. A preferred set of patients is at least 6 months old. Another preferred set of patients is in the age group 2-55 years old, and another preferred set of patients is in the age group 11-55 years old. A further preferred set of patients is less than 11 years old e.g 2-11 years old. In all cases, however, regardless of age, the patient will have been pre-immunised as defined herein.

Where the pre-immunisation antigen is a diphtheria toxoid or tetanus toxoid then the patient will typically have received the toxoid as the ‘D’ or ‘T’ antigen, respectively, in a D-T-P or a D-T pre-immunisation. Such immunizations are typically given to newborn children at ages 2, 3, and 4 months. Where the immunization includes a pertussis vaccine, that vaccine may be a whole cell or cellular pertussis vaccine (‘Pw’), but is preferably an acellular pertussis vaccine (‘Pa’). Pre-immunisation Pa vaccines will generally include one, two or three of the following well-known and well-characterised B.pertussis antigens: (1) pertussis toxoid (‘PT’), detoxified either by chemical means or by site-directed mutagenesis e.g. the ‘9K/129G’ mutant [30], (2) filamentous hemagglutinin (‘FHA’), (3) pertactin (also known as ‘69 kiloDalton outer membrane protein’). Acellular pertussis vaccines may also include agglutinogen 2 and/or agglutinogen 3.

Where the pre-immunisation antigen is a diphtheria toxoid or tetanus toxoid then the patient may also or alternatively have received the toxoid as the carrier protein of a protein-saccharide conjugate. Such conjugates include the ‘PRP-D’ or ‘PRP-T’ Hib conjugates [see Table 14-7 of ref. 32] e.g. the ProHIBIT™ product.

Where the pre-immunisation antigen is CRM197 then the patient will typically have been pre-immunised with a Hib conjugate and/or a multivalent pneumococcal conjugate. Such immunizations are typically given to newborn children at ages 2, 3, and 4 months. Hib conjugates that use a CRM197 carrier include the ‘HbOC’ conjugates [Table 14-7 of ref. 32] e.g the HibTITER™ product. Pneumococcal conjugates that use a CRM197 carrier include the 7-valent PCV7 mixtures e.g the PrevNar™ vaccine [31]. The patient may also have been pre-immunised with a serogroup C meningococcal (‘MenC’) conjugate. MenC conjugates that use CRM197 carrier include Meninvact™/Menjugate™ [5] and Meningitec™. Preferably, however, the patient has been pre-immunised with Hib and/or pneumococcal conjugate, but not with a MenC conjugate. If the patient has been pre-immunised with a MenC conjugate then the vaccine administered according to the invention may or may not include a serogroup C conjugate.

Diphtheria and tetanus toxoids are well known and well characterized proteins [e.g see chapter 13 of ref. 32] that can be obtained by treating the toxin with an inactivating chemical, such as formalin or formaldehyde. CRM197 is also well known and well characterized [33-36], and has been widely used as a carrier in conjugated saccharide vaccines.

### CRM197 and Dt Share Many Carrier Epitopes.

The result of the pre-immunisation is that the patient's immune system has been exposed to the pre-immunisation antigens. For pre-immunisation with diphtheria toxoid (Dt) or tetanus toxoid, this generally means that the patient will have raised an anti-Dt or -Tt antibody response (typically to give an anti-Dt titer>0.01 IU/ml) and will possess memory B and/or T lymphocytes specific for Dt or Tt i.e. pre-immunisation with Dt or Tt is typically adequate to elicit an anamnestic anti-Dt or -Tt immune response in the patient. For pre-immunisation where Dt or Tt (or derivative) is a carrier for a saccharide within a conjugate then the pre-immunisation will have raised an anti-saccharide response and the patient will possess memory B and/or T lymphocytes specific for the saccharide i.e. the pre-immunisation is typically adequate to elicit an anamnestic anti-saccharide immune response in the patient. The pre-immunisation was preferably adequate to elicit protective immunity in the patient e.g. against diphtheria or tetanus disease.

Thus the patients to be immunised according to the invention are distinct from patients in general, as they are members of a subset of the general population whose immune systems have already mounted an immune response to the pre-immunisation antigens, such that immunization according to the invention with a pneumococcal conjugate that includes a diphtheria toxoid and/or tetanus toxoid (or derivative thereof) carrier elicits a different immune response in the subset than in patients who have not previously mounted an immune response to the pre-immunisation antigens. Patients who have been pre-immunised with Dt and/or Tt (or derivative) as the carrier of a conjugate (particularly of a Hib conjugate) are preferred. Particularly preferred patients have been pre-immunised with Dt and/or Tt (or derivative) as the carrier of a conjugate and also with Dt and/or Tt as an unconjugated immunogen.

As well as having been pre-immunised with a diphtheria toxoid and/or tetanus toxoid (or derivative), in conjugated or non-conjugated form, the patient may have been pre-immunised with other antigens. Such antigens include, but are not limited to: pertussis antigen(s)—see above; Haemophilus influenzae type B—see above; hepatitis B surface antigen (HBsAg); poliovirus, such as an inactivated poliovirus vaccine (IPV); meningococcal capsular saccharide conjugates—see above; influenza virus; BCG; hepatitis A virus antigens; measles virus; mumps virus; rubella virus; varicella virus; etc. The patient may or may not have been pre-immunised with one or more pneumococcal capsular saccharide conjugate(s).

In some preferred embodiments, at the time when a patient first receives a pneumococcal conjugate, they have already been pre-immunised with Dt and/or Tt (or derivative). In other embodiments, a pneumococcal conjugate is administered to a patient who has already been pre-immunised with both (i) Dt and/or Tt or a derivative and (ii) a pneumococcal conjugate.

### The Conjugates

The invention immunizes patients with conjugated saccharides. Conjugation is used to enhance the immunogenicity of saccharides, as it converts them from T-independent antigens to T-dependent antigens, thus allowing priming for immunological memory. Conjugation is particularly useful for pediatric vaccines [e.g. ref. 37] and is a well known technique [e.g reviewed in refs. 38 to 46].

Typically the Streptococcus pneumoniae compositions/medicaments of the invention (or in any of the immunogenic compositions of the invention described herein) will comprise conjugated saccharide antigens, wherein the saccharides are derived from at least four serotypes of pneumococcus chosen from the group consisting of 1, 2, 3, 4, 5, 6A, 6B, 7F, 8, 9N, 9V, 10A, 11A, 12F, 14, 15B, 17F, 18C, 19A, 19F, 20, 22F, 23F and 33F. Preferably the four serotypes include 6B, 14, 19F and 23F. More preferably, at least 7 serotypes are included in the composition, for example those derived from serotypes 4, 6B, 9V, 14, 18C, 19F, and 23F. More preferably still more than 7 serotypes are included in the composition, for instance at least 10, 11, 12, 13 or 14 serotypes. For example the composition in one embodiment includes 10 or 11 capsular saccharides derived from serotypes 1, 4, 5, 6B, 7F, 9V, 14, 18C, 19F and 23F, and optionally 3 (all conjugated). In a preferred embodiment of the invention at least 13 saccharide antigens (preferably all conjugated) are included, although further saccharide antigens, for example 23 valent (such as serotypes 1, 2, 3, 4, 5, 6B, 7F, 8, 9N, 9V, 1A, 11A, 12F, 14, 15B, 17F, 18C, 19A, 19F, 20, 22F, 23F and 33F), are also contemplated by the invention. For example, mixtures of polysaccharides from 23 different serotypes are widely used, as are conjugate vaccines with polysaccharides from between 5 and 11 different serotypes [72]. For example, PrevNar™ [31] contains antigens from seven serotypes (4, 6B, 9V, 14, 18C, 19F, and 23F) with each saccharide individually conjugated to CRM197 by reductive amination, with 2 μg of each saccharide per 0.5 ml dose (4 kg of serotype 6B), and with conjugates adsorbed on an aluminium phosphate adjuvant. The composition preferably includes at least serotypes 6B, 14, 19F and 23F.

The capsular saccharides of each of these serotypes are well known. Meningococcal saccharides are also well known (see above for description of the saccharides and envisaged combinations of serogroup saccharides of the invention).

The saccharides used according to the invention may be O-acetylated as (e.g with the same O-acetylation pattern as seen in native capsular saccharides), or they may be partially or totally de-O-acetylated at one or more positions of the saccharide rings, or they may be hyper-O-acetylated relative to the native capsular saccharides.

The saccharides used according to the invention may be shorter than the native capsular saccharides seen in bacteria. Thus the saccharides may be depolymerized, with depolymerization occulting after purification but before conjugation. Depolymerization reduces the chain length of the saccharides. A possible depolymerization method involves the use of hydrogen peroxide [9]. Hydrogen peroxide is added to a saccharide (e.g to give a final H2O2 concentration of 1%), and the mixture is then incubated (e.g at about 55° C.) until a desired chain length reduction has been achieved. Another depolymerization method involves acid hydrolysis [10]. Other depolymerization methods are known to the skilled person. The saccharides used to prepare conjugates for use according to the invention may be obtainable by any of these depolymerization methods. Depolymerization can be used in order to provide an optimum chain length for immunogenicity and/or to reduce chain length for physical manageability of the saccharides.

Typical carrier proteins for use in conjugates are bacterial toxins or toxoids, such as diphtheria toxin (or its CRM97 mutant) and tetanus toxin. Other known carrier proteins include the N.meningitidis outer membrane protein, synthetic peptides, heat shock proteins, pertussis proteins, cytokines, lymphokines, hormones, growth factors, artificial proteins comprising multiple human CD4+ T cell epitopes from various pathogen-derived antigens, protein D from non-typeable H.influenzae, pneumococcal surface protein PspA, iron-uptake proteins, toxin A or B from C.difficile, etc. According to the invention, however, the pnenococcal conjugates include a diphtheria toxoid and/or tetanus toxoid (or derivative thereof, such as CRM197) carrier protein. Covalent conjugation is preferred.

It is possible to use more than one carrier protein in the compositions. Thus different carrier proteins can be used for different serotypes e.g serotype 7F might be conjugated to protein D while serotype 18C saccharide might be conjugated to tetanus toxoid. It is also possible to use more than one carrier protein for a particular saccharide antigen e.g serotype 7F saccharide might be in two groups, with some conjugated to CRM197 and some conjugated to protein D. In general, however, it is preferred to use the same carrier protein for all or the majority of the pneumococcal saccharides in the composition. The saccharide composition can be conjugated to Dt and Tt, Dt and Cnm197, CRM197 and Tt, and Dt, CRM197 and Tt. Protein D may be added to any of these lists of carriers for conjugating (the majority) of the saccharides to. In one embodiment only 1 serotype is conjugated to Dt or CRM197 and/or only one conjugated to Tt.

A single carrier protein might carry more than one saccharide antigen [51]. For example, a single carrier protein might have conjugated to it capsular saccharides from serotypes 7F and 18C. To achieve this goal, saccharides can be mixed prior to the conjugation reaction. In general, however, it is preferred to have separate conjugates for each serotype. Conjugates may be mixed to give substantially a 1:1:1:1 ratio (measured as mass of saccharide) e.g the mass of each serogroup's saccharide is within +10% of each other. A typical quantity of meningococcal antigen per serogroup in a composition is between 1 μg and 20 μg e.g between 2 and 10 μg per serotype.

Conjugates with a saccharide:protein ratio (w/w) of between 1:15 (i.e. excess protein) and 15:1 (i.e. excess saccharide), preferably between 1:10 and 10:1, more preferably between 1:5 and 5:1, may be used.

Conjugates may be used in conjunction with free carrier protein [52]. When a given carrier protein is present in both free and conjugated form in a composition of the invention, however, the unconjugated form is preferably no more than 5% of the total amount of the carrier protein in the composition as a whole, and more preferably present at less than 2% by weight. Similarly, unconjugated saccharide is preferably no more than 15% by weight of the total amount of saccharide.

Any suitable conjugation reaction can be used, with any suitable linker where necessary (see above an references therein for typical conjugation reactions which may be used in the present aspect of the invention). If a linker is employed, in one embodiment it is ADH (adipic acid dihydrazide). Conjugates obtainable by such methods are preferred conjugates for use according to the invention e.g conjugates comprising a diphtheria toxoid and or tetanus toxoid carrier (and optionally an adipic acid linker). Conjugates are preferably prepared separately and then mixed. After mixing, the concentration of the mixed conjugates can be adjusted e.g with sterile pyrogen-free, phosphate-buffered saline. Each conjugate, before mixing, preferably contains no more than 15 μg of carrier.

### Further Antigenic Components of Compositions Used According to the Invention

In addition to pneumococcal conjugates, compositions used according to the invention may optionally include 1, 2 or 3 of the following further antigens:

1. A conjugated capsular saccharide from N. meningitidis; refs. 69-71; see above].

In one embodiment the compositions/medicaments of the invention do not comprise any meningococcal saccharide conjugates. Preferably a tetanus toxid in present as a carrier.

2. A Conjugated Capsular Saccharide from H.influenzue B [e.g chapter 14 of ref. 32].

The carrier protein for the conjugate may be CRM197, Dt, a tetanus toxoid or an outer membrane complex of N.meningitidis. The saccharide moiety of the conjugate may be a polysaccharide (e.g full-length polyribosylribitol phosphate (PRP)), but it is preferred to depolymerize the capsular polysaccharides to form oligosaccharides (e.g. MW from 1 to 5 kDa). A preferred Hib conjugate comprises an oligosaccharide covalently linked to CRM197 or tetanus toxoid via an adipic acid linker [73,74]. Administration of the Hib antigen preferably results in an anti-PRP antibody concentration of >0.15 μg/ml, and more preferably >1 μg/ml. Where a composition includes a Hib saccharide antigen, it preferably does not also include an aluminium hydroxide adjuvant. If the composition includes an aluminium phosphate adjuvant then the Hib antigen may be adsorbed to the adjuvant [75] or it may be non-adsorbed [27]. Prevention of adsorption can be achieved by selecting the correct pH during antigen/adjuvant mixing, an adjuvant with an appropriate point of zero charge, and an appropriate order of mixing for the various different antigens in a composition [76].

3. A protein antigen from Neisseria meningitidis serogroup B [e.g ref. 77].

The composition may comprise one or more of these further antigens. These can be in the form of isolated outer membrane proteins, or in the form of a subunit antigen preparation.

Such antigens may or may not be adsorbed to an aluminium salt.

If pneumococcal conjugates are being administered in a series of doses then none, some or all of the doses may include these extra antigens.

Compositions containing the pneumococcal conjugates in one embodiment do not include meninogoccal capsular saccharide conjugates.

In one embodiment they do not include pertussis antigens. In one embodiment they do not include hepatitis B virus surface antigen. In one embodiment they do not include poliovirus. A composition preferably contains no more than 50 μg of diphtheria toxoid/CRM197 per pneumococcal conjugate, and more preferably no more than 50 μg of diphtheria toxoid/CRM197 for all pneumococcal conjugates combined. A composition preferably contains no more than 50 μg of tetanus toxoid per pneumococcal conjugate, and more preferably no more than 50 μg of tetanus toxoid for all pneumococcal conjugates combined.

### Third Aspect of the Invention

In a third aspect of the invention, the inventors have devised ways to administer various vaccines which use tetanus toxoid and/or DT (and/or CRM197). The written description of the first and second aspects of the invention above and claims 45 onward is also relevant and incorporated by reference to this third aspect of the invention.

There is further provided a method for immunizing a human patient against a disease caused by Neisseria meningitidis, Bordetella pertussis, Clostridium tetani, Corynebacterium diphtheriae and Streptococcus pneumoniae comprising the step of administering to the human patient the following vaccines with the following administration scheme:

| | Visit 1 | Visit 2 | Visit 3 | Visit 4 |
|---|---|---|---|---|
| DTP | X | | X | |
| Strep | | X | | X |
| MenC | | X | | X |

wherein the visit to the medical practitioner all occur in the first 8 months of life,
wherein there is at least 2 weeks between each consecutive visit,
wherein DTP comprises DT, TT, and either whole cell (Pw) or acellular (Pa) pertussis antigens,
wherein Strep is a multivalent pneumococcal capsular saccharide conjugate vaccine comprising at least 7, 10, 11, 13 or 14 conjugated serotypes,
wherein MenC comprises a conjugated N. meningitidis serogroup C capsular saccharide,
wherein at least one conjugated saccharide in each of the Strep and MenC vaccines is conjugated to DT or CRM197, or at least one conjugated saccharide in each of the Strep and MenC vaccines is conjugated to TT.

In a further aspect there is provided a method for immunizing a human patient against a disease caused by Neisseria meningitidis, Bordetella pertussis, Clostridium tetani, Corynebacterium diphtheriae and Streptococcus pneumoniae comprising the step of administering to the human patient the following vaccines with the following administration scheme:

| | Visit 1 | Visit 2 | Visit 3 | Visit 4 |
|---|---|---|---|---|
| DTP | X | X | X | |
| Strep | X | | X | |
| MenC | | X | | Optionally X |

wherein the visit to the medical practitioner all occur in the first 8 months of life,
wherein there is at least 2 weeks between each consecutive visit,
wherein DTP comprises DT, TT, and either whole cell (Pw) or acellular (Pa) pertussis antigens,
wherein Strep is a multivalent pneumococcal capsular saccharide conjugate vaccine comprising at least 7, 10, 11, 13 or 14 conjugated serotypes,
wherein MenC comprises a conjugated N. meningitidis serogroup C capsular saccharide,
wherein at least one conjugated saccharide in each of the Strep and MenC vaccines is conjugated to DT or CRM197, or at least one conjugated saccharide in each of the Strep and MenC vaccines is conjugated to TT.

In a further embodiment there is provided a method for immunizing a human patient against a disease caused by Neisseria meningitidis, Bordetella pertussis, Clostridium tetani, Corynebacterium diphtheriae and Streptococcus pneumoniae comprising the step of administering to the human patient the following vaccines with the following administration scheme:

| | Visit 1 | Visit 2 | Visit 3 |
|---|---|---|---|
| DTP | X | X | X |
| Strep | X | X | |
| MenC | | X | X |

wherein the visit to the medical practitioner all occur in the first 8 months of life,
wherein there is at least 2 weeks between each consecutive visit,
wherein DTP comprises DT, TT, and either whole cell (Pw) or acellular (Pa) pertussis antigens,
wherein Strep is a multivalent pneumococcal capsular saccharide conjugate vaccine comprising at least 7, 10, 11, 13 or 14 conjugated serotypes,
wherein MenC comprises a conjugated N. meningitidis serogroup C capsular saccharide,
wherein at least one conjugated saccharide in each of the Strep and MenC vaccines is conjugated to DT or CRM197, or at least one conjugated saccharide in each of the Strep and MenC vaccines is conjugated to TT.

### General Considerations of the Aspects of the Invention

### The Vaccine Composition

The composition used according to the invention will typically include a pharmaceutically acceptable carrier. Such carriers include any carrier that does not itself induce the production of antibodies; harmful to the individual receiving the composition. Suitable carriers are typically large, slowly metabolised macromolecules such as proteins, polysaccharides, polylactic acids, polyglycolic acids, polymeric amino acids, amino acid copolymers, sucrose, trehalose, lactose, and lipid aggregates (such as oil droplets or liposomes). Such carriers are well known to those of ordinary skill in the art.

The vaccines may also contain diluents, such as water, saline, glycerol, etc. Additionally, auxiliary substances, such as wetting or emulsifying agents, pH buffering substances, and the like, may be present. Sterile pyrogen-free, phosphate-buffered physiologic saline is a typical carrier. A thorough discussion of pharmaceutically acceptable carriers and excipients is available in reference 78.

Compositions used according to the invention may include an antimicrobial, particularly if packaged in a multiple dose format.

Compositions used according to the invention may comprise detergent e.g a Tween (polysorbate), such as Tween 80. Detergents are generally present at low levels e.g <0.01%.

Compositions used according to the invention may include sodium salts (e.g sodium chloride and/or sodium phosphate). These can be used for tonicity. A concentration of 10≅2 mg/ml NaCl is typical e.g. about 8.8 mg/ml. A concentration of 1.2 mg/ml sodium phosphate is typical.

Compositions used according to the invention will generally include a buffer e.g a phosphate buffer.

Compositions used according to the invention may comprise a sugar alcohol (e.g mannitol) or a disaccharide (e.g sucrose or trehalose) eg at about 15-30 mg/ml (e.g 25 mg/ml), particularly if they are to be lyophilised or if they include material which has been reconstituted from lyophilised material. Certain compositions, however, may not be lyophilised i.e. meningococcal or pneumococcal conjugates might be in aqueous form, from the packaging stage to the administration stage. Compositions will generally be administered directly to a patient. Direct delivery may be accomplished by parenteral injection (e.g subcutaneously, intraperitoneally, intravenously, intramuscularly, or to the interstitial space of a tissue), or by rectal, oral, vaginal, topical, transdermal, intranasal, ocular, aural, pulmonary or other mucosal administration. Intramuscular administration (e.g. to the thigh or the upper arm) is preferred. Injection may be via a needle (e.g a hypodermic needle), but needle-free injection may alternatively be used. A typical intramuscular dose is 0.5 ml.

Meningococcal or pneumococcal conjugates from multiple serogroups/serotypes are administered in admixture within a single composition. The composition may be administered as a single dose, or may be administered more than once in a multiple dose schedule. Multiple doses may be used in a primary immunization schedule and/or in a booster immunization schedule. A primary dose schedule may be followed by a booster dose schedule of the meningococcal or pneumococcal conjugates. Suitable timing between priming doses (e.g. between 4-16 weeks), and between priming and boosting, can be routinely determined. The conjugates may conveniently be administered at the same time as other vaccines e.g. at the same time as a D-T-P vaccine, or at the same time as a pneumococcal or meningococcal conjugate vaccine, or at the same time as an influenza vaccine, or at the same time as a MMR or MMRV vaccine. These vaccines will; generally be administered separately but during the same visit to the doctor.

Bacterial infections can affect various areas of the body and so compositions may be prepared in various forms. For example, the compositions may be prepared as injectables, either as liquid solutions or suspensions. Solid forms suitable for solution in, or suspension in, liquid vehicles prior to injection can also be prepared (e.g a lyophilised composition). The composition may be prepared for topical administration e.g as an ointment, cream or powder. The composition be prepared for oral administration e.g as a tablet or capsule, or as a syrup (optionally flavoured). The composition may be prepared for pulmonary administration e.g as an inhaler, using a fine powder or a spray. The composition may be prepared as a suppository or pessary. The composition may be prepared for nasal, aural or ocular administration e.g as spray, drops, gel or powder [e.g refs 79 & 80]. In general, however, the meningococcal or pneumococcal conjugates are formulated for intramuscular injection.

Compositions used according to the invention may or may not include a vaccine adjuvant. Adjuvants which may be used in compositions of the invention include, but are not limited to:

A. Mineral-containing compositions Mineral containing compositions suitable for use as adjuvants in the invention include mineral salts, such as aluminium salts and calcium salts. The invention includes mineral salts such as hydroxides (e.g oxyhydroxides), phosphates, sulphates, etc. [e.g see chapters 8 & 9 of ref. 81], or mixtures of different mineral compounds, with the compounds taking any suitable form (e.g gel, crystalline, amorphous, etc.), and with adsorption being preferred. The mineral containing compositions may also be formulated as a particle of metal salt [82].

Aluminium phosphates may be employed in the compositions of the invention, and a typical adjuvant is amorphous aluminium hydroxyphosphate with PO4/Al molar ratio between 0.84 and 0.92, included at about 0.6 mg Al3+/ml.

Adsorption with a low dose of aluminium phosphate may be used e.g between 50 and 100 μg Al3+ per conjugate per dose. Where a composition includes conjugates from multiple bacterial species then not all conjugates need to be adsorbed;

B. Oil Emulsions Oil emulsion compositions suitable for use as adjuvants in the invention include squalene-water emulsions, such as MF59 [Chapter 10 of ref. 81, see also ref. 83] (5% Squalene, 0.5% Tween 80, and 0.5% Span 85, formulated into submicron particles using a microfluidizer). Complete Freund's adjuvant (CFA) and incomplete Freund's adjuvant (IFA) may also be used.

### C. Saponin Formulations [Chapter 22 of Rep 81]

Saponin formulations may also be used as adjuvants in the invention. Saponins are a heterologous group of sterol glycosides and triterpenoid glycosides that are found in the bark, leaves, stems, roots and even flowers of a wide range of plant species. Saponin from the bark of the Quillaia saponaria Molina tree have been widely studied as adjuvants. Saponin can also be commercially obtained from Smilax ornata (sarsaprilla), Gypsophilla paniculata (brides veil), and Saponaria off cianalis (soap; root). Saponin adjuvant formulations include purified formulations, such as QS21, as well as lipid formulations, such as ISCOMs. QS21 is marketed as Stimulon™.

Saponin compositions have been purified using HPLC and RP-HPLC. Specific purified fractions using these techniques have been identified, including QS7, QS17, QS18, QS21, QH-A, QH-B and QH-C. Preferably, the saponin is QS21. A method of production of QS21 is disclosed in ref. 84.

Saponin formulations may also comprise a sterol, such as cholesterol [85]. Combinations of saponins and cholesterols can be used to form unique particles called immunostimulating complexs (ISCOMs) [chapter 23 of ref. 81]. ISCOMs typically also include a phospholipid such as phosphatidylethanolamine or phosphatidylcholine. Any known saponin can be used in ISCOMs. Preferably, the ISCOM includes one or more of QuilA, QHA & QHC. ISCOMs are further described in refs. 85-87. Optionally, the ISCOMS may be devoid of additional detergent [88].

A review of the development of saponin based adjuvants can be found in refs. 89 & 90.

### D. Virosomes and Virus-Like Particles

Virosomes and virus-like particles (VLPs) can also be used as adjuvants in the invention. These structures generally contain one or more proteins from a virus optionally combined or formulated with a phospholipid. They are generally non-pathogenic, non-replicating and generally do not contain any of the native viral genome. The viral proteins may be recombinantly produced or isolated from whole viruses. These viral proteins suitable for use in virosomes or VLPs include proteins derived from influenza virus (such as HA or NA), Hepatitis B virus (such as core or capsid proteins), Hepatitis E virus, measles virus, Sindbis virus, Rotavirus, Foot-and-Mouth Disease virus, Retrovirus, Norwalk virus, human Papilloma virus, HIV, RNA-phages, QJ3-phage (such as coat proteins), GA-I phage, fr-phage, AP205 phage, and Ty (such as retrotransposon Ty protein pi). VLPs are discussed further in refs. 91-96. Virosomes are discussed further in, for example, ref. 97.

### E. Bacterial or Microbial Derivatives

Adjuvants suitable for use in the invention include bacterial or microbial derivatives such as non-toxic derivatives of enterobacterial lipopolysaccharide (LPS), Lipid A derivatives, immunostimulatory oligonucleotides and ADP-ribosylating toxins and detoxified derivatives thereof.

Non-toxic derivatives of LPS include monophosphoryl lipid A (MPL) and 3-O-deacylated MPL (3dMPL). 3dMPL is a mixture of 3 de-O-acylated monophosphoryl lipid A with 4, 5 or 6 acylated chains. A preferred “small particle” form of 3 De-O-acylated monophosphoryl lipid A is disclosed in ref. 98. Such “small particles” of 3dMPL are small enough to be sterile filtered through a 0.22 μm membrane [98]. Other non-toxic LPS derivatives Include monophosphoryl lipid A mimics, such as aminoalkyl glucosaminide phosphate derivatives e.g RC-529 [99,100].

Lipid A derivatives include derivatives of lipid A from Escherichia cold such as OM-174. OM-174 is described for example in refs. 101 & 102.

Immunostimulatory oligonucleotides suitable for use as adjuvants in the invention include nucleotide; sequences containing a CpG motif (a dinucleotide sequence containing an unmethylated cytosine linked by a phosphate bond to a guanosine). Double-stranded RNAs and oligonucleotides containing palindromic or poly(dG) sequences have also been shown to be immunostimulatory.

The CpG's can include nucleotide modifications/analogs such as phosphorothioate modifications and can be double-stranded or single-stranded. References 103, 104 and 105 disclose possible analog substitutions e.g replacement of guanosine with 2′-deoxy-7-deazaguanosine. The adjuvant effect of CpG oligonucleotides is further discussed in refs. 106-111.

The CpG sequence may be directed to TLR9, such as the motif GTCGTT or TTCGTT [112]. The CpG sequence may be specific for inducing a Th1 immune response, such as a CpG-A ODN, or it may be more specific for inducing a B cell response, such a CpG-B ODN. CpG-A and CpG-B ODNs are discussed in refs. 113-115. Preferably, the CpG is a CpG-A ODN.

Preferably, the CpG oligonucleotide is constructed so that the 5′ end is accessible for receptor recognition. Optionally, two CpG oligonucleotide sequences may be attached at their 3′ ends to form “immunomers”. See, for example, refs. 112 & 116-118.

Bacterial ADP-ribosylating toxins and detoxified derivatives thereof may be used as adjuvants in the invention. Preferably, the protein is derived from E. coli (E. coli heat labile enterotoxin “LT”), cholera (“CT”), or pertussis (“PT”). The use of detoxified ADP-ribosylating toxins as mucosal adjuvants is described in ref. 119 and as parenteral adjuvants in ref. 120. The toxin or toxoid is preferably in the form of a holotoxin, comprising both A and B subunits. Preferably, the A subunit contains a detoxifying mutation; preferably the B subunit is not mutated. Preferably, the adjuvant is a detoxified LT mutant such as LT-K63, LT-R72, and LT-G192. The use of ADP-ribosylating toxins and I detoxified derivatives thereof, particularly LT-K63 and LT-R72, as adjuvants can be found in refs. 121-128. Numerical reference for amino acid substitutions is preferably based on the alignments of the A and B subunits of ADP-ribosylating toxins set forth in ref. 129, specifically incorporated herein by reference in its entirety.

F. Human immunomodulators Human immunomodulators suitable for use as adjuvants in the invention include cytokines, such as interleukins (e.g IL-1, IL-2, IL-4, IL-5, IL-6, IL-7, IL-12 [130], etc.) [131], interferons (e.g interferon-γ), macrophage colony stimulating factor, and tumor necrosis factor.
G. Bioadhesives and Mucoadhesives Bioadhesives and mucoadhesives may also be used as adjuvants in the invention. Suitable bioadhesives include esterified hyaluronic acid microspheres [132] or mucoadhesives such as cross-linked derivatives of poly(acrylic acid), polyvinyl alcohol, polyvinyl pyrollidone, polysaccharides and carboxymethylcellulose. Chitosan and derivatives thereof may also be used as adjuvants in the invention [133].

### H. Microparticles

Microparticles may also be used as adjuvants in the invention. Microparticles (i.e. a particle of 100 nm to 150 nm in diameter, more preferably 200 nm to 30 μm in diameter, and most preferably 500 nm to 10 μm in diameter) formed from materials that are biodegradable and non-toxic (e.g. a poly(a-hydroxy acid), a polyhydroxybutyric acid, a polyorthoester, a polyanhydride, a polycaprolactone, etc.), with poly(lactide-co-glycolide) are preferred, optionally treated to have a negatively-charged surface (e.g with SDS) or a positively-charged surface (e.g with a cationic detergent, such as CTAB).

### I. Liposomes (Chapters 13 & 14 of ref 81)

Examples of liposome formulations suitable for use as adjuvants are described in refs. 134-136.

### J. Polyoxyethylene Ether and Polyoxyethylene Ester Formulations

Adjuvants suitable for use in the invention include polyoxyethylene ethers and polyoxyethylene esters [137]. Such formulations further include polyoxyethylene sorbitan ester surfactants in combination with an octoxynol [138] as well as polyoxyethylene alkyl ethers or ester surfactants in combination with at least one additional non-ionic surfactant such as an octoxynol [139]. Preferred polyoxyethylene ethers are selected from the following group: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, and polyoxyethylene-23-lauryl ether.

### L. Muramylpeptides

Examples of muramyl peptides suitable for use as adjuvants in the invention include N-acetyl muramyl-L-threonyl-D-isoglutamine (thr-MDP), N-acetyl-normuramyl-L-alanyl-D-isoglutamine (nor-MDP), and N-acetylmuramyl-L-alanyl-D-isoglutaminyl-L-alanine-2-(1′-2′-dipalmitoyl-sn-glycero-3-hydroxyphosphoryloxy)-ethylamine MTP-PE).

### K Polyphosphazene (PCPP):

PCPP formulations are described, for example, in refs. 140 and 141.

### M. Imidazoquinolone Compounds.

Examples of imidazoquinolone compounds suitable for use adjuvants in the invention include Imiquamod and its homologues (e.g “Resiquimod 3M”), described further in refs. 142 and 143.

### N. Thiosemicarbazone Compounds.

Examples of thiosemicarbazone compounds, as well as methods of formulating, manufacturing, and screening for compounds all suitable for use as adjuvants in the invention include those described in ref. 144. The thiosemicarbazones are particularly effective in the stimulation of human peripheral blood mononuclear cells for the production of cytokines, such as TNF-α.

### O. Tryptanthrin Compounds.

Examples of tryptanthrin compounds, as well as methods of formulating, manufacturing, and screening for compounds all suitable for use as adjuvants in the invention include those described in ref. 145. The tryptanthrin compounds are particularly effective in the stimulation of human peripheral blood mononuclear cells for the production of cytokines, such as TNF-α.

The invention may also comprise combinations of aspects of one or more of the adjuvants identified above. For example, the following adjuvant compositions may be used in the invention: (1) a saponin and an oil-in-water emulsion [146]; (2) a saponin (e.g QS21)+a non-toxic LPS derivative (e.g. 3dMPL) [147]; (3) a saponin (e.g. QS21)+a non-toxic LPS derivative (e.g 3dMPL)+a cholesterol; (4) a saponin (e.g QS21)+3dMPL+IL-12 (optionally+a sterol) [148]; (5) combinations of 3dMPL with, for example, QS21 and/or oil-in-water emulsions [149]; (6) SAF, containing 10% squalane, 0.4% Tween 80™, 5% pluronic-block polymer L121, and thr-MDP, either microfluidized into a submicron emulsion or vortexed to generate a larger particle size emulsion. (7) Ribi™ adjuvant system (RAS), (Ribi Immunochem) containing 2% squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™); (8) one or more mineral salts (such as an aluminum salt)+a non-toxic derivative of LPS (such as 3dMPL); and (9) one or more mineral salts (such as an aluminum salt)+an immunostimulatory oligonucleotide (such as a nucleotide sequence including a CpG motif).

Other substances that act as immunostimulating agents are disclosed in chapter 7 of ref. 81.

The use of an aluminium hydroxide or aluminium phosphate adjuvant is preferred, and conjugates are generally adsorbed to these salts [e.g examples 7 & 8 of ref. 9; example J of ref. 10]. Mixing with aluminium salts with no adsorption is also possible [27, 76]. Calcium phosphate is another preferred adjuvant. Conjugates may be mixed with (and optionally adsorbed to) the adjuvants separately and then the conjugates may be mixed together, or the conjugates may be mixed together and then mixed with adjuvant.

The pH of compositions used according to the invention is preferably between 6 and 8, preferably about 7. Stable pH may be maintained by the use of a buffer. Where a composition comprises an aluminium hydroxide salt, it is preferred to use a histidine buffer [150]. The composition may be sterile and/or pyrogen-free. Compositions may be isotonic with respect to humans.

Compositions may include a preservative (e.g thiomersal, 2-phenoxyethanol), or may be preservative-free. Preferred compositions of the invention do not include any mercurial material e.g. they are thiomersal-free.

To prevent interference between antigens, particularly conjugate antigens, it is possible to include a polyanionic polymer, such as poly-L-glutamic acid [151].

Compositions may be presented in vials, or they may be presented in ready-filled syringes. The syringes may be supplied with or without needles. A syringe will include a single dose of the composition, whereas a vial may include a single dose or multiple doses. Injectable compositions will usually be liquid solutions or suspensions. Alternatively, they may be presented in solid form (e.g freeze-dried) for solution or suspension in liquid vehicles prior to injection.

Compositions may be packaged in unit dose form or in multiple dose form. For multiple dose forms, vials are preferred to pre-filled syringes. Effective dosage volumes can be routinely established, but a typical human dose of the composition for injection has a volume of 0.5 ml.

Where a composition is to be prepared extemporaneously prior to use (e.g where a component is presented in lyophilised form) and is presented as a kit, the kit may comprise two vials, or it may comprise one ready-filled syringe and one vial, with the contents of the syringe being used to reactivate the contents of the vial prior to injection. For compositions that include a serogroup A capsular saccharide then the serogroup A saccharide may be lyophilised, whereas saccharide(s) from other serogroup(s) may be present in liquid form.

Compositions will comprise an immunologically effective amount of the meningococcal conjugates, as well as any other components, as needed. By ‘immunologically effective amount’, it is meant that the administration of that amount to an individual, either in a single dose or as part of a series, elicits a protective anti-meningococcal or anti-pneumococcal immune response in patients. This amount varies depending upon the health and physical condition of the individual to be treated, age, the taxonomic group of individual to be heated (e.g non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range that can be determined through routine trials, and a typical quantity of each meningococcal antigen per dose is between 1 μg and 20 μg per serogroup/serotype (measured in terms of saccharide) e.g between 2 and 10 μg per serogroup/serotype. A dose of about 4 μg per serogroup/serotype may be used (i.e. a total of 16 μg in a tetravalent MenACWY mixture).

The total amount of carrier protein in a composition preferably does not exceed 100 μg/dose e.g it is ≦90 μg/dose, ≦80 μg/dose, ≦70 μg/dose, ≦60 μg/dose, ≦50 μg Idose, etc. The total amount of carrier protein in a composition will generally be at least 10 μg/dose.

General The term “comprising” encompasses “including” as well as “consisting” e.g a composition “comprising” X may consist exclusively of X or may include something additional eg X+Y. The term “about” in relation to a numerical value x means, for example, x±10%.

The word “substantially” does not exclude “completely” e.g. a composition which is “substantially free” from Y may be completely free from Y. Where necessary, the word “substantially” may be omitted from the definition of the invention.

The term “saccharide” throughout this specification may indicate polysaccharide or oligosaccharide and includes both. Polysaccharides are isolated from bacteria or isolated from bacteria and sized to some degree by known methods (see for example EP497524 and EP497525) and preferably by microfluidization. Polysaccharides can be sized in order to reduce viscosity in polysaccharide samples and/or to improve filterability for conjugated products. Oligosaccharides have a low number of repeat units (typically 5-30 repeat units) and are typically hydrolysed polysaccharides.

MODES FOR CARRYING OUT THE INVENTION

Study No.: 217744/085 (DTPa-Hep B-IPV-085)
Title: A phase III, open labeled, randomized, multicenter, clinical study of the safety and immunogenicity of a
primary series of GlaxoSmithKline Biologicals' (GSK Biologicals') DTaP-HepB-IPV candidate vaccine
coadministered with HibTITER ® and Prevnar ® to healthy infants at 2, 4, and 6 months of age as compared
to the separate administration of Infanrix ® + Engerix-B ® + IPOL ® + HibTITER + Prevnar and to GSK
Biologicals' DTaP-HepB-IPV candidate vaccine coadministered with HibTITER.
Rationale: The present primary vaccination study evaluated the safety and immunogenicity of GSK
Biologicals' DTaP-HepB-IPV combined vaccine (DTaP-HBV-IPV) when co-administered with Haemophilus
influenzae type b Conjugate Vaccine (Hib) and Pneumococcal 7-valent Conjugate Vaccine (PnC) compared
to separate administration of DTaP vaccine (DTaP), Hepatits B Recombinant vaccine (HBV), Poliovirus
Vaccine Inactivated (IPV), Hib and PnC and to DTaP-HBV-IPV when coadministered with Hib with PnC
administered two weeks after each of the DTaP-HBV-IPV doses.
Phase: III
Study Period: Feb. 8, 2002 to Aug. 4, 2003.
Study Design: Open, multicenter, primary vaccination study with three parallel groups. Healthy infants at 2
months of age were randomized to one of the three groups with a balanced allocation (1:1:1).
Centres: 12 centers in the United States.
Indication: DTaP, HBV, IPV, Hib and PnC vaccines are indicated for active immunization of infants in the
first year of life against diphtheria, tetanus, pertussis, hepatitis B, poliomyelitis, Haemophilus influenzae type
b, and Streptococcus pneumoniae serotypes 4, 6B, 9V, 14, 18C, 19F, and 23F diseases.
Treatment: Study groups were as follow:

Combination Vaccine Group: receiving one dose each of DTaP-HBV-IPV + Hib + PnC at 2, 4, 6 months of

age.

Separate Vaccine Group: receiving one dose each of DTaP + HBV + IPV + Hib + PnC at 2, 4, 6 months of

age; HBV was not to be administered at 4 months of age to subjects who received a dose of hepatitis B
vaccine prior to enrollment in this study.

Staggered Vaccine Group: receiving one dose each of DTaP-HBV-IPV + Hib at 2, 4, 6 months of age (PnC

administered 2 weeks following each dose of DTaP-HBV-IPV + Hib) .
All vaccines were to be administered by deep intramuscular injection except for IPV, which was administered
by subcutaneous injection in the left deltoid. DTaP-HBV-IPV or DTaP injections were to be administered in
the upper right anterolateral thigh, and HBV injections were to be administered in the lower right anterolateral
thigh. PnC and Hib injections were to be administered in the upper and lower left anterolateral thigh,
respectively.
Objectives: The primary objective was to demonstrate that the immunogenicity of GSK Biologicals' DTaP-
HBV-IPV combined vaccine co-administered with Hib and PnC as a three dose primary vaccination course is
non-inferior to that of separately administered DTaP, HBV, IPV, Hib and PnC with respect to diphtheria,
tetanus, pertussis, and poliovirus.
Primary Outcome/Efficacy Variable (s):
Criteria for meeting this objective (one month after the third dose):

upper limit of two-sided 90% CI on geometric mean concentration (GMC) ratio (Separate Vaccine Group
over Combination Vaccine Group) below 1.5 for each pertussis antigen
upper limit of two-sided 95% CI on absolute difference (Separate Vaccine Group minus Combination
Vaccine Group) for seroprotection rate below 10% for D and T antigens:

seroprotection status for anti-D and anti-T antibody concentrations ≧0.1 IU/ml

upper limit of two-sided 95% CI on absolute difference (Separate Vaccine Group minus Combination
Vaccine Group) for seroprotection rate below 5% for the three Polio antigens:

seroprotection status for anti-poliovirus type 1, 2 and 3 antibody titers ≧1:8

Secondary Outcome/Efficacy Variable (s):

Immunogenicity one month after the third dose of the primary series of vaccinations:

Vaccine response to PT, FHA and PRN, defined as appearance of antibodies in subjects who were initially

seronegative (i.e., with concentrations < cut-off value) or at least maintenance of pre-vaccination antibody
concentrations in subjects who were initially seropositive (i.e., with concentrations ≧ cut-off value)

Seropositivity status for anti-PT, anti-FHA, anti-PRN (defined as antibody concentrations ≧5 EL.U/ml) .
Seroprotection to hepatitis B (defined as anti-HBs antibody concentrations ≧10 mlU/ml) .
Seroprotection status for anti-PRP (defined as antibody concentrations ≧0.15 μg/ml and ≧1.0 μg/ml) .
Seropositivity status for anti-pneumococcal antibody concentrations to the 7 PnC serotypes (defined as

antibody concentrations ≧0.05 μg/ml) .

Anti-diphtheria, anti-tetanus, anti-HBs and anti-PRP antibody concentrations, and anti-poliovirus types 1, 2

and 3 antibody titers

Safety:

Incidence of fever of any intensity (rectal temperature ≧38.0° C./≧100.4° F.) occurring within 4 days (days 0-

day 3) after each vaccine dose

Incidence of grade 2 or grade 3 fever (>38.5° C./>101.3° F.) occurring within four days after each vaccine

dose

Incidence of grade 3 fever (>39.5° C./>103.1° F.) occurring within four days after each vaccine dose
Incidence of fever >39.0° C. (>102.2° F.) occurring within four days after each vaccine dose
Incidence of other solicited general symptoms (any intensity, grade 2 or grade 3 and grade 3) occurring

within four days after each vaccine dose

Incidence of solicited local symptoms (any intensity, grade 2 or grade 3 and grade 3) occurring within four

days after each vaccine dose

Incidence of antipyretic use within four days after each vaccine dose
Incidence of antibiotic use within four days after each vaccine dose
Incidence of unsolicited adverse events (AEs) occurring throughout the entire active phase of the study

(including the 31-day follow-up period after the last dose of DTaP/DTaP-HBV-IPV)

Occurrence of SAEs throughout the entire study including the six month (182 day) follow-up period after the

last dose of DTaP/DTaP-HBV-IPV.

Extended safety follow-up period up to six months after the last dose of study vaccine (five months after
end of active phase)

Statistical methods:
The analyses were performed on the Total vaccinated cohort, the According-To-Protocol (ATP) cohort and
the Extended Safety Follow-up (ESFU) cohort,

The Total vaccinated cohort includes all subjects having received at least one vaccine dose and for whom

data for endpoint measures were available. In the Total vaccinated cohort, subjects were analyzed according
to the vaccine(s) that were actually administered.

The ATP cohort for immunogenicity includes subjects for whom assay results were available for antibodies

against at least one study vaccine antigen one month after the 3-dose primary vaccination.

The ESFU cohort includes all subjects for whom follow-up data were available beyond the 31-day period

(Day 0-Day 30) following the last dose of vaccine (from Day 31 post last dose to the last study contact),
and includes those subjects who dropped out before the scheduled 5-month follow-up contact at study
completion (six months post last dose of study vaccine).
Analysis of Immunogenicity:
The immunogenicity analyses were based on the ATP cohort.
GMCs/GMTs and seropositivity/seroprotection rates were calculated with their 95% CI for each group, at
each blood sampling time point.
Standardized asymptotic 95% Confidence Intervals (CIs) for the difference in pertussis vaccine response
rates and in Seroprotection rates of D, T, poliovirus types 1, 2 and 3 (Separate Vaccine Group minus
Combination Vaccine Group) one month after the third dose were computed. For each pertussis antigen one
month after the third vaccine dose, the 90% CIs of the GMC/GMT ratios (Separate Vaccine Group divided by
Combination Vaccine Group) were computed using an ANCOVA model on the logarithm10 transformation of
the concentrations/titers. The ANCOVA model included the vaccine group as fixed effect (all three groups)
and the pre-vaccination concentration/titer as regressor.
The primary objective was reached if the upper limits of the 90% CIs for the GMC/GMT ratios and of the
asymptotic 95% Cis for the differences in seroprotection rates for all primary endpoints were all below the
clinical limits defining non-inferiority
Analysis of Safety:
For solicited symptoms, the analyses were performed on the total vaccinated cohort. For each solicited
symptom, the percentage of subjects with the symptom and its exact 95% Confidence Interval (CI) was
summarized by vaccine group, by dose and across doses.
The percentage of subjects using antibiotics and antipyretics within the four days post-vaccination was
summarized with their 95% CI.
The percentage of subjects reporting unsolicited symptoms during the active phase (from dose 1 to day 30
following last vaccine dose) was summarized by vaccine group according to the WHO preferred term and
reported to the total vaccinated cohort.
The percentage of subjects reporting unsolicited symptoms during the Extended Safety Follow-up Period
(occurring from day 31 after last vaccine dose to last study contact) was summarized by vaccine group
according to the WHO preferred term and reported to the ESFU cohort.
SAEs reported during the entire study including the Extended Safety Follow-up Period (ESFU) were also
described and reported to the total vaccinated cohort.
Study Population: Healthy male or female infants born after a normal gestation period of 36-42 weeks, 6-
12 weeks of age at the time of the first primary vaccination, and free of obvious health problems as
established by medical history and clinical examination before entering into the study. The subjects were not
to have been previously vaccinated against diphtheria, tetanus, pertussis, poliomyelitis, Haemophilus
influenzae type b, and/or Streptococcus pneumoniae disease; more than one previous dose of hepatitis B
vaccine at least 30 days prior to enrollment, or have had a history of these diseases. Subjects' parents/
guardians provided written informed consent.

Number of Subjects
(Modified ITT Cohort)

| | Separate | Staggered | Combination |
|---|---|---|---|
| | Vaccine Group | Vaccine Group | Vaccine Group |
| Planned, N | 190 | 190 | 190 |
| Randomized, N | 188 | 188 | 199 |
| Completed, n (%) | 172 (91.5) | 177 (94.1) | 183 (92.0) |
| Total Number Subjects Withdrawn, N (%) | 16 (8.5) | 11 (5.9) | 16 (8.0) |
| Withdrawn due to Adverse Events, n (%) | 0 (0.0) | 0 (0.0) | 0 (0.0) |
| Withdrawn due to Lack of Efficacy, n (%) | Not applicable | Not applicable | Not applicable |
| Withdrawn for other reasons, n (%) | 16 (8.5) | 11 (5.9) | 16 (8.0) |

Demographics

| | | | |
|---|---|---|---|
| Group | | | |
| Total Vaccinated Cohort, N | 188 | 188 | 199 |
| Females:Males | 95:93 | 97:91 | 104:95 |
| Mean age, weeks (SD) | 8.6 (1.24) | 8.8 (1.41) | 8.6 (1.22) |
| Race: White, n (%) | 157 (83.5) | 160 (85.1) | 167 (83.9) |

Primary Efficacy Results:
Differences in response rates between the Separate Vaccine Group and Combination
Vaccine Group with their 95% CIs one month following the third DTaP/DTaP-HB-IPV
vaccination for anti-diphteria, anti-tetanus and the three Polio antigens - ATP cohort

| | | | | | | | Separate minus | | |
|---|---|---|---|---|---|---|---|---|---|
| | Separate | | | Combination | | | Combination Vaccine | | |
| | Vaccine Group | | | Vaccine Group | | | Group | | |
| | | | Rate | | | Rate | Diff in | 95% CI | |
| Endpoint | N | n | (%) | N | n | (%) | rates (%) | LL | UL |
| anti-D ≧0.1 IU/ml | 155 | 153 | 98.7 | 168 | 167 | 99.4 | −0.70 | −4.05 | 2.12* |
| anti-T ≧0.1 IU/ml | 155 | 152 | 98.1 | 168 | 168 | 100.0 | −1.94 | −5.54 | 0.32* |
| anti-poliovirus 1 ≧ 1:8 | 153 | 153 | 100.0 | 168 | 168 | 100.0 | 0.00 | −2.45 | 2.24** |
| anti-polio virus 2 ≧ 1:8 | 153 | 153 | 100.0 | 168 | 168 | 100.0 | 0.00 | −2.45 | 2.24** |
| anti-poliovirus 3 ≧ 1:8 | 153 | 153 | 100.0 | 168 | 168 | 100.0 | 0.00 | −2.45 | 2.24** |

N = number of subjects with available results
n = number of subjects with concentration/titer above the specified cut-off or with a vaccine response
% = n/N × 100
95% CI = 95% standardized asymptotic confidence interval; LL = lower limit; UL = upper limit
*non-inferiority criterion met: upper limit below the 10% difference clinical limit for non-inferiority
**non-inferiority criterion met: upper limit below the 5% difference clinical limit for non-inferiority

Primary Efficacy Results:
Ratios of adjusted GMCs/GMTs between Separate Vaccine Group and Combination Vaccine Group
with their 90% CIs one month following the third vaccination for each Pertussis antigen - ATP cohort

| | Separate | | Combination | | Separate/Combination | | |
|---|---|---|---|---|---|---|---|
| | Vaccine Group | | Vaccine Group | | Vaccine Group | | |
| | | Adjusted | | Adjusted | Ratio of adjusted | 90% CI | |
| Endpoint | N | GMC/GMT | N | GMC/GMT | GMC/GMT | LL | UL |
| anti-PT concentration | 142 | 28.6 | 155 | 48.1 | 0.59 | 0.51 | 0.70* |
| anti-FHA concentration | 141 | 97.6 | 154 | 111.9 | 0.87 | 0.76 | 1.00* |
| anti-PRN concentration | 142 | 80.6 | 156 | 95.3 | 0.85 | 0.72 | 0.99* |

N: number of subjects with both pre- and post-vaccination results available
90% CI = 90% confidence interval; LL = Lower Limit; UL = Upper Limit (Ancova model including all 3 vaccine groups: adjustment for baseline antibody concentrations/titers)
*non-inferiority criterion met: upper limit below the 1.5 fold clinical limit for non-inferiority

Secondary Outcome Variable (s):
Vaccine response rates for anti-PT, anti-FHA and anti-PRN
antibodies one month following the third DTaP/DTaP-HBV-IPV
vaccination by pre-vaccination seropositivity status-ATP cohort

| | | Pre-vaccination | | Responders | | | |
|---|---|---|---|---|---|---|---|
| Anti- | | seropositivity | | | | 95% CI | |
| body | Group | status | N | n | % | LL | UL |
| anti-PT | Sepa- | Seropositive | 14 | 10 | 71.4 | 41.9 | 91.6 |
| | rate | Seronegative | 128 | 125 | 97.7 | 93.3 | 99.5 |
| | | Total | 142 | 135 | 95.1 | 90.1 | 98.0 |
| | Stag- | Seropositive | 16 | 14 | 87.5 | 61.7 | 98.4 |
| | gered | Seronegative | 136 | 134 | 98.5 | 94.8 | 99.8 |
| | | Total | 152 | 148 | 97.4 | 93.4 | 99.3 |
| | Combi- | Seropositive | 18 | 18 | 100 | 81.5 | 100 |
| | nation | Seronegative | 137 | 135 | 98.5 | 94.8 | 99.8 |
| | | Total | 155 | 153 | 98.7 | 95.4 | 99.8 |
| anti-FHA | Sepa- | Seropositive | 86 | 81 | 94.2 | 87.0 | 98.1 |
| | rate | Seronegative | 55 | 55 | 100 | 93.5 | 100 |
| | | Total | 141 | 136 | 96.5 | 91.9 | 98.8 |
| | Stag- | Seropositive | 92 | 89 | 96.7 | 90.8 | 99.3 |
| | gered | Seronegative | 61 | 61 | 100 | 94.1 | 100 |
| | | Total | 153 | 150 | 98.0 | 94.4 | 99.6 |
| | Combi- | Seropositive | 95 | 93 | 97.9 | 92.6 | 99.7 |
| | nation | Seronegative | 59 | 59 | 100 | 93.9 | 100 |
| | | Total | 154 | 152 | 98.7 | 95.4 | 99.8 |
| anti-PRN | Sepa- | Seropositive | 66 | 59 | 89.4 | 79.4 | 95.6 |
| | rate | Seronegative | 76 | 76 | 100 | 95.3 | 100 |
| | | Total | 142 | 135 | 95.1 | 90.1 | 98.0 |
| | Stag- | Seropositive | 71 | 62 | 87.3 | 77.3 | 94.0 |
| | gered | Seronegative | 83 | 83 | 100 | 95.7 | 100 |
| | | Total | 154 | 145 | 94.2 | 89.2 | 97.3 |
| | Combi- | Seropositive | 85 | 72 | 84.7 | 75.3 | 91.6 |
| | nation | Seronegative | 71 | 71 | 100 | 94.9 | 100 |
| | | Total | 156 | 143 | 91.7 | 86.2 | 95.5 |

N = number of subjects with both pre- and post-vaccination results available
n = number of responders
% = n/N × 100
95% CI = exact 95% confidence interval; LL = lower limit, UL = upper limit
Total = subjects either seropositive or seronegative at pre-vaccination
Vaccine response to PT, FHA and PRN is defined as appearance of antibodies in subjects who were initially seronegative (i.e., with concentrations < cut-off value) or at least maintenance of pre-vaccination antibody concentrations in subjects who were initially seropositive (i.e., with concentrations < cut-off value).

Secondary Outcome Variable (s):
Seropositivity rates and GMCs for anti-PT, anti-FHA and anti-PRN
antibody concentrations prior to vaccination and one month
following the third DTaP/DTaP-HBV-IPV vaccination - ATP cohort

| | | | | ≧5 EL.U/ml | | | | GMC | | |
|---|---|---|---|---|---|---|---|---|---|---|
| | | | | | | 95% CI | | | 95% CI | |
| Antibody | Group | Timing | N | n | % | LL | UL | Value | LL | UL |
| anti-PT | Separate | PRE | 143 | 15 | 10.5 | 6.0 | 16.7 | 2.9 | 2.7 | 3.1 |
| | | PIII | 155 | 151 | 97.4 | 93.5 | 99.3 | 28.9 | 25.2 | 33.2 |
| | Staggered | PRE | 155 | 17 | 11.0 | 6.5 | 17.0 | 2.9 | 2.7 | 3.2 |
| | | PIII | 158 | 155 | 98.1 | 94.6 | 99.6 | 50.3 | 44.6 | 56.7 |
| | Combination | PRE | 156 | 18 | 11.5 | 7.0 | 17.6 | 2.8 | 2.7 | 3.0 |
| | | PIII | 168 | 166 | 98.8 | 95.8 | 99.9 | 48.7 | 42.8 | 55.4 |
| anti-FHA | Separate | PRE | 142 | 87 | 61.3 | 52.7 | 69.3 | 6.5 | 5.6 | 7.6 |
| | | PIII | 155 | 154 | 99.4 | 96.5 | 100.0 | 96.4 | 85.5 | 108.7 |
| | Staggered | PRE | 156 | 94 | 60.3 | 52.1 | 68.0 | 6.7 | 5.7 | 7.9 |
| | | PIII | 158 | 158 | 100.0 | 97.7 | 100.0 | 117.7 | 105.9 | 130.9 |
| | Combination | PRE | 155 | 95 | 61.3 | 53.1 | 69.0 | 6.8 | 5.8 | 8.0 |
| | | PIII | 168 | 168 | 100.0 | 97.8 | 100.0 | 113.7 | 101.5 | 127.3 |
| anti-PRN | Separate | PRE | 143 | 67 | 46.9 | 38.5 | 55.4 | 5.3 | 4.5 | 6.2 |
| | | PIII | 155 | 153 | 98.7 | 95.4 | 99.8 | 79.1 | 66.8 | 93.6 |
| | Staggered | PRE | 157 | 72 | 45.9 | 37.9 | 54.0 | 5.5 | 4.7 | 6.4 |
| | | PIII | 158 | 158 | 100.0 | 97.7 | 100.0 | 99.5 | 87.5 | 113.2 |
| | Combination | PRE | 157 | 85 | 54.1 | 46.0 | 62.1 | 6.4 | 5.4 | 7.5 |
| | | PIII | 168 | 168 | 100.0 | 97.8 | 100.0 | 93.7 | 82.3 | 106.7 |

N = number of subjects with available results
n = number of subjects with concentration above the specified cut-off
% = n/N × 100
95% CI = 95% confidence interval; LL = Lower Limit; UL = Upper Limit
PRE = immediately before the first vaccination
PIII = approximately one month following the third DTaP/DTaP-HBV-IPV vaccination (approximately 7 months of age)

Secondary Outcome Variable (s):
Seroprotection rates and GMCs for anti-HBs antibody prior to vaccination
and one month following the third DTaP/DTaP-HBV-IPV vaccination-ATP cohort

| | | | | ≧10 mIU/ml | | | | GMC | | |
|---|---|---|---|---|---|---|---|---|---|---|
| | | | | | | 95% CI | | | 95% CI | |
| Antibody | Group | Timing | N | n | % | LL | UL | Value | LL | UL |

Total subjects regardless of hepatitis B vaccination status prior to study entry

| | | | | | | | | | | |
|---|---|---|---|---|---|---|---|---|---|---|
| anti-HBs | Separated | PRE | 141 | 16 | 11.3 | 6.6 | 17.8 | 6.7 | 5.7 | 7.9 |
| | | PIII | 154 | 152 | 98.7 | 95.4 | 99.8 | 667.5 | 534.1 | 834.3 |
| | Staggered | PRE | 151 | 26 | 17.2 | 11.6 | 24.2 | 8.8 | 7.1 | 11.0 |
| | | PIII | 158 | 158 | 100.0 | 97.7 | 100.0 | 1515.3 | 1250.6 | 1835.9 |
| | Combination | PRE | 150 | 34 | 22.7 | 16.2 | 30.2 | 10.1 | 7.9 | 13.0 |
| | | PIII | 167 | 164 | 98.2 | 94.8 | 99.6 | 1123.6 | 912.0 | 1384.2 |

N = number of subjects with available results
n = number of subjects with concentration above the specified cut-off
% = n/N × 100
95% CI = 95% confidence interval; LL = Lower Limit; UL = Upper Limit
PRE = immediately before the first vaccination
PIII = approximately one month following the third DTaP/DTaP-HBV-IPV vaccination (approximately 7 months of age)

Secondary Outcome Variable (s):
Seroprotection rates and GMCs for anti-PRP antibody concentrations prior to
vaccination and one month following the third DTaP/DTaP-HBV-IPV vaccination - ATP cohort

| | | | ≧0.15 μg/ml | | | | ≧1 μg/ml | | | | GMC | | |
|---|---|---|---|---|---|---|---|---|---|---|---|---|---|
| | | | | | 95% CI | | | | 95% CI | | | 95% CI | |
| Group | Timing | N | n | % | LL | UL | n | % | LL | UL | Value | LL | UL |
| Separate | PRE | 143 | 101 | 70.6 | 62.4 | 77.9 | 13 | 9.1 | 4.9 | 15.0 | 0.247 | 0.209 | 0.293 |
| | PIII | 155 | 154 | 99.4 | 96.5 | 100.0 | 141 | 91.0 | 85.3 | 95.0 | 9.246 | 7.378 | 11.587 |
| Staggered | PRE | 156 | 103 | 66.0 | 58.0 | 73.4 | 15 | 9.6 | 5.5 | 15.4 | 0.215 | 0.184 | 0.252 |
| | PIII | 158 | 158 | 100.0 | 97.7 | 100.0 | 150 | 94.9 | 90.3 | 97.8 | 8.384 | 7.020 | 10.013 |
| Combination | PRE | 157 | 107 | 68.2 | 60.3 | 75.4 | 14 | 8.9 | 5.0 | 14.5 | 0.224 | 0.193 | 0.261 |
| | PIII | 168 | 168 | 100.0 | 97.8 | 100.0 | 161 | 95.8 | 91.6 | 98.3 | 9.619 | 7.995 | 11.572 |

N = number of subjects with available results
n = number of subjects with concentration above the specified cut-off
% = n/N × 100
95% CI = 95% confidence interval; LL = Lower Limit; UL = Upper Limit
PRE = immediately before the first vaccination
PIII = approximately one month following the third DTaP/DTaP-HBV-IPV vaccination (approximately 7 months of age)

Secondary Outcome Variable (s):
Seropositivity rates and GMCs for serotypes 4, 6B, 9V, 14, 18C, 19F, and 23F anti-S.
pneumoniae antibodies prior to vaccination and following the third PnC vaccination - ATP cohort

| anti- | | | | ≧0.05 μg/ml | | | | GMC | | |
|---|---|---|---|---|---|---|---|---|---|---|
| S.pneumoniae | | | | | | 95% CI | | | 95% CI | |
| serotype | Group | Timing | N | n | % | LL | UL | Value | LL | UL |
| 4 | Separate | PRE | 128 | 38 | 29.7 | 21.9 | 38.4 | 0.04 | 0.04 | 0.05 |
| | | PIII | 156 | 156 | 100.0 | 97.7 | 100.0 | 2.07 | 1.81 | 2.37 |
| | Staggered | PRE | 141 | 39 | 27.7 | 20.5 | 35.8 | 0.04 | 0.04 | 0.05 |
| | | PIIIP | 155 | 155 | 100.0 | 97.6 | 100.0 | 1.62 | 1.44 | 1.83 |
| | Combination | PRE | 144 | 47 | 32.6 | 25.1 | 40.9 | 0.04 | 0.04 | 0.05 |
| | | PIII | 164 | 163 | 99.4 | 96.6 | 100.0 | 1.74 | 1.54 | 1.98 |
| 6B | Separate | PRE | 132 | 69 | 52.3 | 43.4 | 61.0 | 0.07 | 0.06 | 0.09 |
| | | PIII | 148 | 134 | 90.5 | 84.6 | 94.7 | 0.67 | 0.52 | 0.87 |
| | Staggered | PRE | 142 | 75 | 52.8 | 44.3 | 61.2 | 0.08 | 0.06 | 0.09 |
| | | PIIIP | 154 | 148 | 96.1 | 91.7 | 98.6 | 0.59 | 0.49 | 0.72 |
| | Combination | PRE | 146 | 82 | 56.2 | 47.7 | 64.4 | 0.10 | 0.08 | 0.13 |
| | | PIII | 163 | 156 | 95.7 | 91.4 | 98.3 | 0.80 | 0.65 | 0.99 |
| 9V | Separate | PRE | 126 | 63 | 50.0 | 41.0 | 59.0 | 0.07 | 0.06 | 0.09 |
| | | PIII | 151 | 151 | 100.0 | 97.6 | 100.0 | 1.60 | 1.39 | 1.85 |
| | Staggered | PRE | 135 | 81 | 60.0 | 51.2 | 68.3 | 0.08 | 0.07 | 0.10 |
| | | PIIIP | 150 | 150 | 100.0 | 97.6 | 100.0 | 1.11 | 0.97 | 1.28 |
| | Combination | PRE | 138 | 77 | 55.8 | 47.1 | 64.2 | 0.08 | 0.06 | 0.10 |
| | | PIII | 163 | 163 | 100.0 | 97.8 | 100.0 | 1.55 | 1.36 | 1.77 |
| 14 | Separate | PRE | 130 | 121 | 93.1 | 87.3 | 96.8 | 0.46 | 0.35 | 0.61 |
| | | PIII | 156 | 156 | 100.0 | 97.7 | 100.0 | 6.32 | 5.39 | 7.41 |
| | Staggered | PRE | 141 | 129 | 91.5 | 85.6 | 95.5 | 0.38 | 0.30 | 0.48 |
| | | PIIIP | 154 | 154 | 100.0 | 97.6 | 100.0 | 4.51 | 3.91 | 5.19 |
| | Combination | PRE | 146 | 137 | 93.8 | 88.6 | 97.1 | 0.60 | 0.46 | 0.77 |
| | | PIII | 166 | 166 | 100.0 | 97.8 | 100.0 | 4.68 | 4.04 | 5.43 |
| 18C | Separate | PRE | 129 | 75 | 58.1 | 49.1 | 66.8 | 0.09 | 0.07 | 0.12 |
| | | PIII | 153 | 153 | 100.0 | 97.6 | 100.0 | 2.96 | 2.53 | 3.47 |
| | Staggered | PRE | 139 | 85 | 61.2 | 52.5 | 69.3 | 0.09 | 0.07 | 0.11 |
| | | PIIIP | 156 | 156 | 100.0 | 97.7 | 100.0 | 2.37 | 2.06 | 2.72 |
| | Combination | PRE | 143 | 90 | 62.9 | 54.5 | 70.9 | 0.11 | 0.09 | 0.14 |
| | | PIII | 167 | 166 | 99.4 | 96.7 | 100.0 | 2.63 | 2.31 | 3.00 |
| 19F | Separate | PRE | 131 | 101 | 77.1 | 68.9 | 84.0 | 0.18 | 0.14 | 0.23 |
| | | PIII | 147 | 147 | 100.0 | 97.5 | 100.0 | 1.05 | 0.91 | 1.22 |
| | Staggered | PRE | 140 | 112 | 80.0 | 72.4 | 86.3 | 0.19 | 0.15 | 0.24 |
| | | PIIIP | 149 | 149 | 100.0 | 97.6 | 100.0 | 0.75 | 0.66 | 0.86 |
| | Combination | PRE | 143 | 118 | 82.5 | 75.3 | 88.4 | 0.27 | 0.21 | 0.34 |
| | | PIII | 161 | 160 | 99.4 | 96.6 | 100.0 | 1.09 | 0.95 | 1.25 |
| 23F | Separate | PRE | 128 | 66 | 51.6 | 42.6 | 60.5 | 0.07 | 0.06 | 0.09 |
| | | PIII | 146 | 141 | 96.6 | 92.2 | 98.9 | 1.81 | 1.45 | 2.25 |
| | Staggered | PRE | 137 | 81 | 59.1 | 50.4 | 67.4 | 0.09 | 0.07 | 0.11 |
| | | PIIIP | 153 | 150 | 98.0 | 94.4 | 99.6 | 1.29 | 1.09 | 1.53 |
| | Combination | PRE | 140 | 79 | 56.4 | 47.8 | 64.8 | 0.11 | 0.08 | 0.14 |
| | | PIII | 162 | 158 | 97.5 | 93.8 | 99.3 | 1.48 | 1.23 | 1.79 |

N = number of subjects with available results
n = number of subjects with concentration above the specified cut-off
% = n/N × 100
95% CI = 95% confidence interval; LL = Lower Limit; UL = Upper Limit
PRE = immediately before the first vaccination
PIII = approximately one month following the third PnC vaccination for subjects in the Combination Vaccine Group and Separate Vaccine Group (approximately 7 months of age)
PIIIP = approximately two weeks following the third PnC vaccination for subjects in the Staggered Vaccine Group (approximately 7 months of age)

Secondary Outcome Variable (s):
Seroprotection rates and GMCs for anti-diphtheria and
anti-tetanus antibody concentrations prior to vaccination and one
month following the third DTaP/DTaP-HBV-IPV vaccination - ATP cohort

| | | | | ≧0.1 IU/ml | | | | GMC | | |
|---|---|---|---|---|---|---|---|---|---|---|
| | | | | | | 95% CI | | | 95% CI | |
| Antibody | Group | Timing | N | n | % | LL | UL | Value | LL | UL |
| anti-D | Separate | PRE | 143 | 75 | 52.4 | 43.9 | 60.9 | 0.117 | 0.100 | 0.137 |
| | | PIII | 155 | 153 | 98.7 | 95.4 | 99.8 | 1.564 | 1.354 | 1.806 |
| | Staggered | PRE | 157 | 76 | 48.4 | 40.4 | 56.5 | 0.111 | 0.095 | 0.128 |
| | | PIII | 157 | 157 | 100.0 | 97.7 | 100.0 | 3.238 | 2.889 | 3.630 |
| | Combination | PRE | 157 | 64 | 40.8 | 33.0 | 48.9 | 0.096 | 0.083 | 0.111 |
| | | PIII | 168 | 167 | 99.4 | 96.7 | 100.0 | 1.987 | 1.761 | 2.242 |
| anti-T | Separate | PRE | 143 | 134 | 93.7 | 88.4 | 97.1 | 0.583 | 0.489 | 0.696 |
| | | PIII | 155 | 152 | 98.1 | 94.4 | 99.6 | 1.346 | 1.166 | 1.554 |
| | Staggered | PRE | 157 | 142 | 90.4 | 84.7 | 94.6 | 0.561 | 0.468 | 0.672 |
| | | PIII | 158 | 158 | 100.0 | 97.7 | 100.0 | 2.240 | 2.017 | 2.487 |
| | Combination | PRE | 157 | 143 | 91.1 | 85.5 | 95.0 | 0.527 | 0.443 | 0.628 |
| | | PIII | 168 | 168 | 100.0 | 97.8 | 100.0 | 2.428 | 2.180 | 2.704 |

N = number of subjects with available results
n = number of subjects with concentration above the specified cut-off
% = n/N × 100
95% CI = 95% confidence interval; LL = Lower Limit; UL = Upper Limit
PRE = immediately before the first vaccination
PIII = approximately one month following the third DTaP/DTaP-HBV-IPV vaccination (approximately 7 months of age)

Secondary Outcome Variable (s):
Seroprotection rates and GMTs for anti-poliovirus type 1, anti-poliovirus
type 2 and anti-poliovirus type 3 antibody titers prior to vaccination and one
month following the third DTaP/DTaP-HBV-IPV vaccination - Total vaccinated cohort

| | | | | ≧1:8 | | | | GMT | | |
|---|---|---|---|---|---|---|---|---|---|---|
| | | | | | | 95% CI | | | 95% CI | |
| Antibody | Group | Timing | N | n | % | LL | UL | Value | LL | UL |
| anti-poliovirus | Separate | PRE | 143 | 115 | 80.4 | 73.0 | 86.6 | 22.0 | 17.8 | 27.2 |
| 1 | | PIII | 153 | 153 | 100.0 | 97.6 | 100.0 | 225.0 | 191.3 | 264.7 |
| | Staggered | PRE | 152 | 120 | 78.9 | 71.6 | 85.1 | 20.7 | 16.9 | 25.3 |
| | | PIII | 156 | 156 | 100.0 | 97.7 | 100.0 | 793.1 | 660.3 | 952.7 |
| | Combination | PRE | 153 | 121 | 79.1 | 71.8 | 85.2 | 20.7 | 16.7 | 25.5 |
| | | PIII | 168 | 168 | 100.0 | 97.8 | 100.0 | 678.0 | 568.1 | 809.2 |
| anti-poliovirus | Separate | PRE | 143 | 100 | 69.9 | 61.7 | 77.3 | 13.8 | 11.5 | 16.6 |
| 2 | | PIII | 153 | 153 | 100.0 | 97.6 | 100.0 | 228.1 | 198.1 | 262.7 |
| | Staggered | PRE | 152 | 106 | 69.7 | 61.8 | 76.9 | 13.5 | 11.3 | 16.0 |
| | | PIII | 156 | 156 | 100.0 | 97.7 | 100.0 | 608.8 | 500.8 | 740.2 |
| | Combination | PRE | 153 | 124 | 81.0 | 73.9 | 86.9 | 15.0 | 12.7 | 17.8 |
| | | PIII | 168 | 168 | 100.0 | 97.8 | 100.0 | 578.3 | 479.3 | 697.8 |
| anti-poliovirus | Separate | PRE | 143 | 39 | 27.3 | 20.2 | 35.3 | 6.0 | 5.2 | 6.9 |
| 3 | | PIII | 153 | 153 | 100.0 | 97.6 | 100.0 | 454.2 | 390.6 | 528.2 |
| | Staggered | PRE | 152 | 37 | 24.3 | 17.8 | 32.0 | 5.8 | 5.2 | 6.6 |
| | | PIII | 156 | 156 | 100.0 | 97.7 | 100.0 | 1167.5 | 973.9 | 1399.6 |
| | Combination | PRE | 153 | 49 | 32.0 | 24.7 | 40.0 | 6.8 | 5.8 | 8.1 |
| | | PIII | 168 | 168 | 100.0 | 97.8 | 100.0 | 1269.2 | 1061.3 | 1517.8 |

N = number of subjects with available results
n = number of subjects with titer above the specified cut-off
% = n/N × 100
95% CI = 95% confidence interval; LL = Lower Limit; UL = Upper Limit
PRE = immediately before the first vaccination
PIII = approximately one month following the third DTaP/DTaP-HBV-IPV vaccination (approximately 7 months of age)

Secondary Outcome Variable (s):
Incidence of fever, excluding doses with no temperature recording or tympanic
measurements, by intensity and causal relationship reported within four days following
DTaP/DTaP-HBV-IPV vaccination (Day 0-Day 3), by dose and across doses- Total vaccinated cohort

| | | Separate Vaccine Group | | | | Staggered Vaccine Group | | | | Combination Vaccine Group | | | |
|---|---|---|---|---|---|---|---|---|---|---|---|---|---|
| Rectal | | | | 95% CI | | | | 95% CI | | | | 95% CI | |
| temperature* | | n | % | LL | UL | n | % | LL | UL | n | % | LL | UL |
| Dose 1 | | N = 187 | | | | N = 185 | | | | N = 197 | | | |
| ≧38.0° | C. | 40 | 21.4 | 15.7 | 28.0 | 35 | 18.9 | 13.5 | 25.3 | 63 | 32.0 | 25.5 | 39.0 |
| >38.5° | C. | 9 | 4.8 | 2.2 | 8.9 | 3 | 1.6 | 0.3 | 4.7 | 12 | 6.1 | 3.2 | 10.4 |
| >39° | C. | 2 | 1.1 | 0.1 | 3.8 | 2 | 1.1 | 0.1 | 3.9 | 4 | 2.0 | 0.6 | 5.1 |
| >39.5° | C. | 0 | 0.0 | 0.0 | 2.0 | 0 | 0.0 | 0.0 | 2.0 | 1 | 0.5 | 0.0 | 2.8 |
| Causally | | 33 | 17.6 | 12.5 | 23.9 | 33 | 17.8 | 12.6 | 24.1 | 62 | 31.5 | 25.1 | 38.5 |
| related | | | | | | | | | | | | | |
| Dose 2 | | N = 180 | | | | N = 181 | | | | N = 192 | | | |
| ≧38.0° | C. | 59 | 32.8 | 26.0 | 40.2 | 49 | 27.1 | 20.7 | 34.2 | 82 | 42.7 | 35.6 | 50.0 |
| >38.5° | C. | 13 | 7.2 | 3.9 | 12.0 | 10 | 5.5 | 2.7 | 9.9 | 21 | 10.9 | 6.9 | 16.2 |
| >39° | C. | 1 | 0.6 | 0.0 | 3.1 | 7 | 3.9 | 1.6 | 7.8 | 9 | 4.7 | 2.2 | 8.7 |
| >39.5° | C. | 0 | 0.0 | 0.0 | 2.0 | 0 | 0.0 | 0.0 | 2.0 | 2 | 1.0 | 0.1 | 3.7 |
| Causally | | 55 | 30.6 | 23.9 | 37.8 | 46 | 25.4 | 19.2 | 32.4 | 77 | 40.1 | 33.1 | 47.4 |
| related | | | | | | | | | | | | | |
| Dose 3 | | N = 176 | | | | N = 180 | | | | N = 190 | | | |
| ≧38.0° | C. | 53 | 30.1 | 23.4 | 37.5 | 41 | 22.8 | 16.9 | 29.6 | 68 | 35.8 | 29.0 | 43.0 |
| >38.5° | C. | 12 | 6.8 | 3.6 | 11.6 | 9 | 5.0 | 2.3 | 9.3 | 21 | 11.1 | 7.0 | 16.4 |
| >39° | C. | 6 | 3.4 | 1.3 | 7.3 | 3 | 1.7 | 0.3 | 4.8 | 8 | 4.2 | 1.8 | 8.1 |
| >39.5° | C. | 1 | 0.6 | 0.0 | 3.1 | 1 | 0.6 | 0.0 | 3.1 | 2 | 1.1 | 0.1 | 3.8 |
| Causally | | 47 | 26.7 | 20.3 | 33.9 | 35 | 19.4 | 13.9 | 26.0 | 63 | 33.2 | 26.5 | 40.3 |
| related | | | | | | | | | | | | | |
| Across doses | | N = 188 | | | | N = 187** | | | | N = 197** | | | |
| ≧38.0° | C. | 101 | 53.7 | 46.3 | 61.0 | 87 | 46.5 | 39.2 | 53.9 | 134 | 68.0 | 61.0 | 74.5 |
| >38.5° | C. | 30 | 16.0 | 11.0 | 22.0 | 21 | 11.2 | 7.1 | 16.7 | 45 | 22.8 | 17.2 | 29.3 |
| >39° | C. | 9 | 4.8 | 2.2 | 8.9 | 12 | 6.4 | 3.4 | 10.9 | 20 | 10.2 | 6.3 | 15.2 |
| >39.5° | C. | 1 | 0.5 | 0.0 | 2.9 | 1 | 0.5 | 0.0 | 2.9 | 5 | 2.5 | 0.8 | 5.8 |

| | | | | | | | | | | | | |
|---|---|---|---|---|---|---|---|---|---|---|---|---|
| Causally | 94 | 50.0 | 42.6 | 57.4 | 82 | 43.9 | 36.6 | 51.3 | 130 | 66.0 | 58.9 | 72.6 |
| related | | | | | | | | | | | | |

N = number of subjects with a symptom sheet completed
n/% = number/percentage of subjects reporting the specified symptom
95% CI = 95% confidence interval (exact method for proportion); LL = lower limit, UL = upper limit
*Fever (rectal route): temperature ≧38.0° C. (≧100.4° F.). Axillary recordings were increased by 1° C. to derive equivalent rectal temperatures and are included.
**post vaccination information not available for one subject in staggered vaccine group and two subjects in combination vaccine group

Secondary Outcome Variable (s):
Incidence of solicited general symptoms, excluding fever, reported within
four days following DTaP/DTaP-HBV-IPV vaccination (Day 0-Day 3), by dose- Total vaccinated cohort

| | Separate Vaccine Group | | | | Staggered Vaccine Group | | | | Combination Vaccine Group | | | |
|---|---|---|---|---|---|---|---|---|---|---|---|---|
| | | | 95% CI | | | | 95% CI | | | | 95% CI | |
| Intensity | n | % | LL | UL | n | % | LL | UL | n | % | LL | UL |
| Dose 1 | N = 187 | | | | N = 187 | | | | N = 198 | | | |
| Drowsiness | | | | | | | | | | | | |
| Any | 110 | 58.8 | 51.4 | 66.0 | 107 | 57.2 | 49.8 | 64.4 | 123 | 62.1 | 55.0 | 68.9 |
| Grade 2 or 3 | 34 | 18.2 | 12.9 | 24.5 | 28 | 15.0 | 10.2 | 20.9 | 37 | 18.7 | 13.5 | 24.8 |
| Grade 3 | 4 | 2.1 | 0.6 | 5.4 | 5 | 2.7 | 0.9 | 6.1 | 4 | 2.0 | 0.6 | 5.1 |
| Causally | 107 | 57.2 | 49.8 | 64.4 | 103 | 55.1 | 47.7 | 62.3 | 120 | 60.6 | 53.4 | 67.5 |
| related | | | | | | | | | | | | |
| Irritability | | | | | | | | | | | | |
| Any | 116 | 62.0 | 54.7 | 69.0 | 119 | 63.6 | 56.3 | 70.5 | 134 | 67.7 | 60.7 | 74.1 |
| Grade 2 or 3 | 39 | 20.9 | 15.3 | 27.4 | 35 | 18.7 | 13.4 | 25.1 | 46 | 23.2 | 17.5 | 29.7 |
| Grade 3 | 2 | 1.1 | 0.1 | 3.8 | 6 | 3.2 | 1.2 | 6.9 | 10 | 5.1 | 2.4 | 9.1 |
| Causally | 113 | 60.4 | 53.0 | 67.5 | 114 | 61.0 | 53.6 | 68.0 | 132 | 66.7 | 59.6 | 73.2 |
| related | | | | | | | | | | | | |
| Loss of appetite | | | | | | | | | | | | |
| Any | 56 | 29.9 | 23.5 | 37.1 | 56 | 29.9 | 23.5 | 37.1 | 64 | 32.3 | 25.9 | 39.3 |
| Grade 2 or 3 | 9 | 4.8 | 2.2 | 8.9 | 12 | 6.4 | 3.4 | 10.9 | 13 | 6.6 | 3.5 | 11.0 |
| Grade 3 | 1 | 0.5 | 0.0 | 2.9 | 1 | 0.5 | 0.0 | 2.9 | 0 | 0.0 | 0.0 | 1.8 |
| Causally | 56 | 29.9 | 23.5 | 37.1 | 55 | 29.4 | 23.0 | 36.5 | 61 | 30.8 | 24.5 | 37.7 |
| related | | | | | | | | | | | | |
| Dose 2 | N = 180 | | | | N = 183 | | | | N = 192 | | | |
| Drowsiness | | | | | | | | | | | | |
| Any | 104 | 57.8 | 50.2 | 65.1 | 71 | 38.8 | 31.7 | 46.3 | 103 | 53.6 | 46.3 | 60.9 |
| Grade 2 or 3 | 25 | 13.9 | 9.2 | 19.8 | 17 | 9.3 | 5.5 | 14.5 | 28 | 14.6 | 9.9 | 20.4 |
| Grade 3 | 3 | 1.7 | 0.3 | 4.8 | 0 | 0.0 | 0.0 | 2.0 | 5 | 2.6 | 0.9 | 6.0 |
| Causally | 99 | 55.0 | 47.4 | 62.4 | 68 | 37.2 | 30.1 | 44.6 | 96 | 50.0 | 42.7 | 57.3 |
| related | | | | | | | | | | | | |
| Irritability | | | | | | | | | | | | |
| Any | 113 | 62.8 | 55.3 | 69.9 | 103 | 56.3 | 48.8 | 63.6 | 148 | 77.1 | 70.5 | 82.8 |
| Grade 2 or 3 | 41 | 22.8 | 16.9 | 29.6 | 34 | 18.6 | 13.2 | 25.0 | 68 | 35.4 | 28.7 | 42.6 |
| Grade 3 | 8 | 4.4 | 1.9 | 8.6 | 4 | 2.2 | 0.6 | 5.5 | 9 | 4.7 | 2.2 | 8.7 |
| Causally | 110 | 61.1 | 53.6 | 68.3 | 101 | 55.2 | 47.7 | 62.5 | 143 | 74.5 | 67.7 | 80.5 |
| related | | | | | | | | | | | | |
| Loss of appetite | | | | | | | | | | | | |
| Any | 50 | 27.8 | 21.4 | 34.9 | 44 | 24.0 | 18.0 | 30.9 | 53 | 27.6 | 21.4 | 34.5 |
| Grade 2 or 3 | 6 | 3.3 | 1.2 | 7.1 | 8 | 4.4 | 1.9 | 8.4 | 10 | 5.2 | 2.5 | 9.4 |
| Grade 3 | 0 | 0.0 | 0.0 | 2.0 | 0 | 0.0 | 0.0 | 2.0 | 0 | 0.0 | 0.0 | 1.9 |
| Causally | 46 | 25.6 | 19.4 | 32.6 | 43 | 23.5 | 17.6 | 30.3 | 52 | 27.1 | 20.9 | 34.0 |
| related | | | | | | | | | | | | |
| Dose 3 | N = 177 | | | | N = 180 | | | | N = 191 | | | |
| Drowsiness | | | | | | | | | | | | |
| Any | 84 | 47.5 | 39.9 | 55.1 | 61 | 33.9 | 27.0 | 41.3 | 90 | 47.1 | 39.9 | 54.5 |
| Grade 2 or 3 | 19 | 10.7 | 6.6 | 16.3 | 12 | 6.7 | 3.5 | 11.4 | 23 | 12.0 | 7.8 | 17.5 |
| Grade 3 | 1 | 0.6 | 0.0 | 3.1 | 1 | 0.6 | 0.0 | 3.1 | 1 | 0.5 | 0.0 | 2.9 |
| Causally | 77 | 43.5 | 36.1 | 51.1 | 55 | 30.6 | 23.9 | 37.8 | 85 | 44.5 | 37.3 | 51.9 |
| related | | | | | | | | | | | | |
| Irritability | | | | | | | | | | | | |
| Any | 104 | 58.8 | 51.1 | 66.1 | 99 | 55.0 | 47.4 | 62.4 | 131 | 68.6 | 61.5 | 75.1 |
| Grade 2 or 3 | 34 | 19.2 | 13.7 | 25.8 | 29 | 16.1 | 11.1 | 22.3 | 48 | 25.1 | 19.1 | 31.9 |
| Grade 3 | 4 | 2.3 | 0.6 | 5.7 | 7 | 3.9 | 1.6 | 7.8 | 10 | 5.2 | 2.5 | 9.4 |
| Causally | 101 | 57.1 | 49.4 | 64.5 | 96 | 53.3 | 45.8 | 60.8 | 127 | 66.5 | 59.3 | 73.1 |
| related | | | | | | | | | | | | |
| Loss of appetite | | | | | | | | | | | | |
| Any | 53 | 29.9 | 23.3 | 37.3 | 40 | 22.2 | 16.4 | 29.0 | 65 | 34.0 | 27.3 | 41.2 |
| Grade 2 or 3 | 12 | 6.8 | 3.6 | 11.5 | 8 | 4.4 | 1.9 | 8.6 | 13 | 6.8 | 3.7 | 11.4 |
| Grade 3 | 1 | 0.6 | 0.0 | 3.1 | 1 | 0.6 | 0.0 | 3.1 | 3 | 1.6 | 0.3 | 4.5 |
| Causally | 49 | 27.7 | 21.2 | 34.9 | 34 | 18.9 | 13.5 | 25.4 | 61 | 31.9 | 25.4 | 39.1 |
| related | | | | | | | | | | | | |
| Across doses | N = 188 | | | | N = 187* | | | | N = 198* | | | |
| Drowsiness | | | | | | | | | | | | |
| Any | 145 | 77.1 | 70.5 | 82.9 | 131 | 70.1 | 62.9 | 76.5 | 159 | 80.3 | 74.1 | 85.6 |
| Grade 2 or 3 | 59 | 31.4 | 24.8 | 38.5 | 46 | 24.6 | 18.6 | 31.4 | 63 | 31.8 | 25.4 | 38.8 |
| Grade 3 | 8 | 4.3 | 1.9 | 8.2 | 6 | 3.2 | 1.2 | 6.9 | 9 | 4.5 | 2.1 | 8.5 |
| Causally | 139 | 73.9 | 67.0 | 80.1 | 128 | 68.4 | 61.3 | 75.0 | 154 | 77.8 | 71.3 | 83.4 |
| related | | | | | | | | | | | | |
| Irritability | | | | | | | | | | | | |
| Any | 157 | 83.5 | 77.4 | 88.5 | 158 | 84.5 | 78.5 | 89.4 | 185 | 93.4 | 89.0 | 96.5 |
| Grade 2 or 3 | 86 | 45.7 | 38.5 | 53.2 | 72 | 38.5 | 31.5 | 45.9 | 109 | 55.1 | 47.8 | 62.1 |
| Grade 3 | 14 | 7.4 | 4.1 | 12.2 | 14 | 7.5 | 4.2 | 12.2 | 25 | 12.6 | 8.3 | 18.1 |
| Causally | 155 | 82.4 | 76.2 | 87.6 | 155 | 82.9 | 76.7 | 88.0 | 181 | 91.4 | 86.6 | 94.9 |
| related | | | | | | | | | | | | |
| Loss of appetite | | | | | | | | | | | | |
| Any | 101 | 53.7 | 46.3 | 61.0 | 87 | 46.5 | 39.2 | 53.9 | 116 | 58.6 | 51.4 | 65.5 |
| Grade 2 or 3 | 22 | 11.7 | 7.5 | 17.2 | 26 | 13.9 | 9.3 | 19.7 | 28 | 14.1 | 9.6 | 19.8 |
| Grade 3 | 2 | 1.1 | 0.1 | 3.8 | 2 | 1.1 | 0.1 | 3.8 | 3 | 1.5 | 0.3 | 4.4 |
| Causally | 96 | 51.1 | 43.7 | 58.4 | 86 | 46.0 | 38.7 | 53.4 | 112 | 56.6 | 49.4 | 63.6 |
| related | | | | | | | | | | | | |

N = number of subjects with a symptom sheet completed
n/% = number/percentage of subjects reporting the specified symptom
95% CI = 95% confidence interval (exact method for proportion); LL = lower limit, UL = upper limit
Drowsiness Grade 2 = Drowsiness that interfered with normal activity
Drowsiness Grade 3 = Drowsiness that prevented normal activity
Irritability Grade 2 = Crying more than usual/interfered with normal activity
Irritability Grade 3 = Crying that could not be comforted/prevented normal activity
Loss of appetite Grade 2 = Eating less than usual/interfered with normal activity
Loss of appetite Grade 3 = Not eating at all
*post vaccination information not available for one subject in each group

Secondary Outcome Variable (s):
Incidence of solicited local symptoms (any intensity, grade 2 or grade 3 and grade 3) ocurring within four days
following DTaP/DTaP-HBV-IPV vaccination (Day 0-Day 3), by dose and across doses- Total vaccinated cohort

| | | Separate Vaccine Group | | | | Staggered Vaccine Group | | | | Combination Vaccine Group | | | |
|---|---|---|---|---|---|---|---|---|---|---|---|---|---|
| | | | | 95% CI | | | | 95% CI | | | | 95% CI | |
| Symptom | Intensity | n | % | LL | UL | n | % | LL | UL | n | % | LL | UL |
| Dose 1 | | N = 186 | | | | N = 187 | | | | N = 198 | | | |
| Pain | Any | 86 | 46.2 | 38.9 | 53.7 | 72 | 38.5 | 31.5 | 45.9 | 89 | 44.9 | 37.9 | 52.2 |
| | Grade 2 or | 29 | 15.6 | 10.7 | 21.6 | 24 | 12.8 | 8.4 | 18.5 | 38 | 19.2 | 14.0 | 25.4 |
| | 3 | | | | | | | | | | | | |
| | Grade 3 | 6 | 3.2 | 1.2 | 6.9 | 8 | 4.3 | 1.9 | 8.3 | 8 | 4.0 | 1.8 | 7.8 |
| Redness | Any | 76 | 40.9 | 33.7 | 48.3 | 56 | 29.9 | 23.5 | 37.1 | 72 | 36.4 | 29.7 | 43.5 |
| | Grade 2 or | 21 | 11.3 | 7.1 | 16.7 | 17 | 9.1 | 5.4 | 14.2 | 20 | 10.1 | 6.3 | 15.2 |
| | 3 | | | | | | | | | | | | |
| | Grade 3 | 3 | 1.6 | 0.3 | 4.6 | 3 | 1.6 | 0.3 | 4.6 | 3 | 1.5 | 0.3 | 4.4 |
| Swelling | Any | 45 | 24.2 | 18.2 | 31.0 | 33 | 17.6 | 12.5 | 23.9 | 43 | 21.7 | 16.2 | 28.1 |
| | Grade 2 or | 17 | 9.1 | 5.4 | 14.2 | 14 | 7.5 | 4.2 | 12.2 | 21 | 10.6 | 6.7 | 15.8 |
| | 3 | | | | | | | | | | | | |
| | Grade 3 | 2 | 1.1 | 0.1 | 3.8 | 4 | 2.1 | 0.6 | 5.4 | 8 | 4.0 | 1.8 | 7.8 |
| Dose 2 | | N = 180 | | | | N = 183 | | | | N = 192 | | | |
| Pain | Any | 72 | 40.0 | 32.8 | 47.6 | 61 | 33.3 | 26.6 | 40.7 | 84 | 43.8 | 36.6 | 51.1 |
| | Grade 2 or | 29 | 16.1 | 11.1 | 22.3 | 16 | 8.7 | 5.1 | 13.8 | 34 | 17.7 | 12.6 | 23.9 |
| | 3 | | | | | | | | | | | | |
| | Grade 3 | 2 | 1.1 | 0.1 | 4.0 | 4 | 2.2 | 0.6 | 5.5 | 8 | 4.2 | 1.8 | 8.0 |
| Redness | Any | 83 | 46.1 | 38.7 | 53.7 | 68 | 37.2 | 30.1 | 44.6 | 97 | 50.5 | 43.2 | 57.8 |
| | Grade 2 or | 28 | 15.6 | 10.6 | 21.7 | 26 | 14.2 | 9.5 | 20.1 | 43 | 22.4 | 16.7 | 29.0 |
| | 3 | | | | | | | | | | | | |
| | Grade 3 | 6 | 3.3 | 1.2 | 7.1 | 12 | 6.6 | 3.4 | 11.2 | 10 | 5.2 | 2.5 | 9.4 |
| Swelling | Any | 48 | 26.7 | 20.4 | 33.8 | 45 | 24.6 | 18.5 | 31.5 | 69 | 35.9 | 29.2 | 43.2 |
| | Grade 2 or | 10 | 5.6 | 2.7 | 10.0 | 17 | 9.3 | 5.5 | 14.5 | 28 | 14.6 | 9.9 | 20.4 |
| | 3 | | | | | | | | | | | | |
| | Grade 3 | 3 | 1.7 | 0.3 | 4.8 | 5 | 2.7 | 0.9 | 6.3 | 6 | 3.1 | 1.2 | 6.7 |
| Dose 3 | | N = 177 | | | | N = 180 | | | | N = 191 | | | |
| Pain | Any | 71 | 40.1 | 32.8 | 47.7 | 54 | 30.0 | 23.4 | 37.3 | 77 | 40.3 | 33.3 | 47.6 |
| | Grade 2 or | 25 | 14.1 | 9.4 | 20.1 | 8 | 4.4 | 1.9 | 8.6 | 33 | 17.3 | 12.2 | 23.4 |
| | 3 | | | | | | | | | | | | |
| | Grade 3 | 1 | 0.6 | 0.0 | 3.1 | 2 | 1.1 | 0.1 | 4.0 | 6 | 3.1 | 1.2 | 6.7 |
| Redness | Any | 89 | 50.3 | 42.7 | 57.9 | 77 | 42.8 | 35.4 | 50.4 | 96 | 50.3 | 43.0 | 57.6 |
| | Grade 2 or | 37 | 20.9 | 15.2 | 27.6 | 27 | 15.0 | 10.1 | 21.1 | 31 | 16.2 | 11.3 | 22.2 |
| | 3 | | | | | | | | | | | | |
| | Grade 3 | 9 | 5.1 | 2.4 | 9.4 | 10 | 5.6 | 2.7 | 10.0 | 6 | 3.1 | 1.2 | 6.7 |
| Swelling | Any | 55 | 31.1 | 24.3 | 38.5 | 45 | 25.0 | 18.9 | 32.0 | 70 | 36.6 | 29.8 | 43.9 |
| | Grade 2 or | 13 | 7.3 | 4.0 | 12.2 | 13 | 7.2 | 3.9 | 12.0 | 22 | 11.5 | 7.4 | 16.9 |
| | 3 | | | | | | | | | | | | |
| | Grade 3 | 1 | 0.6 | 0.0 | 3.1 | 2 | 1.1 | 0.1 | 4.0 | 5 | 2.6 | 0.9 | 6.0 |
| Across doses | | N = 188 | | | | N = 187* | | | | N = 198* | | | |
| Pain | Any | 118 | 62.8 | 55.4 | 69.7 | 98 | 52.4 | 45.0 | 59.7 | 126 | 63.6 | 56.5 | 70.3 |
| | Grade 2 or | 58 | 30.9 | 24.3 | 38.0 | 35 | 18.7 | 13.4 | 25.1 | 73 | 36.9 | 30.1 | 44.0 |
| | 3 | | | | | | | | | | | | |
| | Grade 3 | 8 | 4.3 | 1.9 | 8.2 | 10 | 5.3 | 2.6 | 9.6 | 18 | 9.1 | 5.5 | 14.0 |
| Redness | Any | 125 | 66.5 | 59.3 | 73.2 | 102 | 54.5 | 47.1 | 61.8 | 135 | 68.2 | 61.2 | 74.6 |
| | Grade 2 or | 62 | 33.0 | 26.3 | 40.2 | 48 | 25.7 | 19.6 | 32.6 | 67 | 33.8 | 27.3 | 40.9 |
| | 3 | | | | | | | | | | | | |
| | Grade 3 | 14 | 7.4 | 4.1 | 12.2 | 20 | 10.7 | 6.7 | 16.0 | 16 | 8.1 | 4.7 | 12.8 |
| Swelling | Any | 84 | 44.7 | 37.4 | 52.1 | 70 | 37.4 | 30.5 | 44.8 | 102 | 51.5 | 44.3 | 58.7 |
| | Grade 2 or | 29 | 15.4 | 10.6 | 21.4 | 30 | 16.0 | 11.1 | 22.1 | 45 | 22.7 | 17.1 | 29.2 |
| | 3 | | | | | | | | | | | | |
| | Grade 3 | 5 | 2.7 | 0.9 | 6.1 | 9 | 4.8 | 2.2 | 8.9 | 16 | 8.1 | 4.7 | 12.8 |

N = number of subjects with a symptom sheet completed
n/% = number/percentage of subjects reporting the specified symptom
95% CI = 95% confidence interval (exact method for proportion); LL = lower limit, UL = upper limit
Pain Grade 2 = Cried/protested on touch
Pain Grade 3 = Cried when limb was moved/spontaneously painful
Redness or Swelling Grade 2 = >5 mm to <20 mm
Redness or Swelling Grade 3 = >20 mm
*post vaccination information not available for one subject in each group

Secondary Outcome Variable (s): Concomitant medication reported within four days
following DTaP/DTaP-HBV-IPV vaccination (Day 0-Day 3) - Total vaccinated cohort

| | Separate Vaccine Group | | | | Staggered Vaccine Group | | | | Combination Vaccine Group | | | |
|---|---|---|---|---|---|---|---|---|---|---|---|---|
| | | | 95% CI | | | | 95% CI | | | | 95% CI | |
| | n | % | LL | UL | n | % | LL | UL | n | % | LL | UL |
| Dose 1 | N = 187 | | | | N = 187 | | | | N = 198 | | | |
| Any medication | 91 | 48.7 | 41.3 | 56.1 | 78 | 41.7 | 34.6 | 49.1 | 91 | 46.0 | 38.9 | 53.2 |
| Any Antibiotic | 2 | 1.1 | 0.1 | 3.8 | 2 | 1.1 | 0.1 | 3.8 | 1 | 0.5 | 0.0 | 2.8 |
| Any Antipyretic | 79 | 42.2 | 35.1 | 49.7 | 70 | 37.4 | 30.5 | 44.8 | 84 | 42.4 | 35.4 | 49.6 |
| Antipyretic | 24 | 12.8 | 8.4 | 18.5 | 12 | 6.4 | 3.4 | 10.9 | 15 | 7.6 | 4.3 | 12.2 |
| Prophylactic | | | | | | | | | | | | |
| Antipyretic for fever | 20 | 10.7 | 6.7 | 16.0 | 25 | 13.4 | 8.8 | 19.1 | 39 | 19.7 | 14.4 | 25.9 |
| Dose 2 | N = 180 | | | | N = 183 | | | | N = 192 | | | |
| Any medication | 83 | 46.1 | 38.7 | 53.7 | 60 | 32.8 | 26.0 | 40.1 | 101 | 52.6 | 45.3 | 59.8 |
| Any Antibiotic | 1 | 0.6 | 0.0 | 3.1 | 1 | 0.5 | 0.0 | 3.0 | 2 | 1.0 | 0.1 | 3.7 |
| Any Antipyretic | 78 | 43.3 | 36.0 | 50.9 | 51 | 27.9 | 21.5 | 35.0 | 91 | 47.4 | 40.2 | 54.7 |
| Antipyretic | 10 | 5.6 | 2.7 | 10.0 | 5 | 2.7 | 0.9 | 6.3 | 8 | 4.2 | 1.8 | 8.0 |
| Prophylactic | | | | | | | | | | | | |
| Antipyretic for fever* | 25 | 13.9 | 9.2 | 19.8 | 18 | 9.8 | 5.9 | 15.1 | 47 | 24.5 | 18.6 | 31.2 |
| Dose 3 | N = 177 | | | | N = 180 | | | | N = 191 | | | |
| Any medication | 73 | 41.2 | 33.9 | 48.9 | 57 | 31.7 | 24.9 | 39.0 | 89 | 46.6 | 39.4 | 53.9 |
| Any Antibiotic | 4 | 2.3 | 0.6 | 5.7 | 9 | 5.0 | 2.3 | 9.3 | 6 | 3.1 | 1.2 | 6.7 |
| Any Antipyretic | 66 | 37.3 | 30.1 | 44.9 | 43 | 23.9 | 17.9 | 30.8 | 75 | 39.3 | 32.3 | 46.6 |
| Antipyretic | 5 | 2.8 | 0.9 | 6.5 | 2 | 1.1 | 0.1 | 4.0 | 5 | 2.6 | 0.9 | 6.0 |
| Prophylactic | | | | | | | | | | | | |
| Antipyretic for fever | 26 | 14.7 | 9.8 | 20.8 | 11 | 6.1 | 3.1 | 10.7 | 37 | 19.4 | 14.0 | 25.7 |
| Across doses | N = 188 | | | | N = 187* | | | | N = 198* | | | |
| Any medication | 136 | 72.3 | 65.4 | 78.6 | 123 | 65.8 | 58.5 | 72.5 | 156 | 78.8 | 72.4 | 84.3 |
| Any Antibiotic | 6 | 3.2 | 1.2 | 6.8 | 11 | 5.9 | 3.0 | 10.3 | 9 | 4.5 | 2.1 | 8.5 |
| Any Antipyretic | 129 | 68.6 | 61.5 | 75.2 | 104 | 55.6 | 48.2 | 62.9 | 145 | 73.2 | 66.5 | 79.3 |
| Antipyretic | 34 | 18.1 | 12.9 | 24.3 | 17 | 9.1 | 5.4 | 14.2 | 25 | 12.6 | 8.3 | 18.1 |
| Prophylactic | | | | | | | | | | | | |
| Antipyretic for fever | 53 | 28.2 | 21.9 | 35.2 | 46 | 24.6 | 18.6 | 31.4 | 89 | 44.9 | 37.9 | 52.2 |

N = number of subjects with at least one medication screen completed
n/% = number/percentage of subjects for whom the specified concomitant medication was given
95% CI = 95% confidence interval (exact method for proportion); LL = lower limit, UL = upper limit
*post vaccination information not available for one subject in each group

Secondary Outcome Variable (s):
Number (%) of subjects with unsolicited adverse events
during the extended follow-up period beyond 31
days following last vaccine dose (ESFU cohort)

| Most frequent 10 | Separate | Staggered | Combination |
|---|---|---|---|
| unsolicited Adverse | Vaccine Group | Vaccine Group | Vaccine Group |
| Events | N = 182 | N = 184 | N = 194 |
| Subjects with any | 154 (84.6) | 164 (89.1) | 165 (85.1) |
| AE(s), n (%) | | | |
| upper respiratory | 78 (42.9) | 85 (46.2) | 84 (43.3) |
| tract infection | | | |
| otitis media | 46 (25.3) | 48 (26.1) | 56 (28.9) |
| rhinitis | 38 (20.9) | 30 (16.3) | 30 (15.5) |
| injection site | 24 (13.2) | 33 (17.9) | 27 (13.9) |
| reaction | | | |
| infection viral | 14 (7.7) | 24 (13.0) | 24 (12.4) |
| tooth ache | 25 (13.7) | 21 (11.4) | 14 (7.2) |
| diarrhea | 23 (12.6) | 15 (8.2) | 13 (6.7) |
| vomiting | 14 (7.7) | 21 (11.4) | 10 (5.2) |
| conjunctivitis | 10 (5.5) | 20 (10.9) | 11 (5.7) |
| coughing | 12 (6.6) | 13 (7.1) | 15 (7.7) |
| moniliasis | 9 (4.9) | 14 (7.6) | 12 (6.2) |
| constipation | 9 (4.9) | 12 (6.5) | 12 (6.2) |
| fever | 10 (5.5) | 14 (7.6) | 9 (4.6) |
| dermatitis | 4 (2.2) | 17 (9.2) | 10 (5.2) |
| contact | | | |
| rash | 10 (5.5) | 10 (5.4) | 10 (5.2) |
| pneumonia | 7 (3.8) | 5 (2.7) | 14 (7.2) |

Safety Results: Number (%) of subjects with unsolicited Adverse
Events occurring within the active phase from dose 1 to day
30 following last vaccine dose (Total vaccinated cohort)

| Most frequent | Separate | Staggered | Combination |
|---|---|---|---|
| unsolicited Adverse | Vaccine Group | Vaccine Group | Vaccine Group |
| Events | N = 188 | N = 187* | N = 198* |
| Subjects with any | 154 (81.9) | 164 (87.7) | 165 (83.3) |
| AE(s), n (%) | | | |
| upper respiratory | 78 (41.5) | 85 (45.5) | 84 (42.4) |
| tract infection | | | |
| otitis media | 46 (24.5) | 48 (25.7) | 56 (28.3) |
| rhinitis | 38 (20.2) | 30 (16.0) | 30 (15.2) |
| injection site | 24 (12.8) | 33 (17.6) | 27 (13.6) |
| reaction | | | |
| infection viral | 14 (7.4) | 24 (12.8) | 24 (12.1) |
| tooth ache | 25 (13.3) | 21 (11.2) | 14 (7.1) |
| diarrhea | 23 (12.2) | 15 (8.0) | 13 (6.6) |
| vomiting | 14 (7.4) | 21 (11.2) | 10 (5.1) |
| conjunctivitis | 10 (5.3) | 20 (10.7) | 11 (5.6) |
| coughing | 12 (6.4) | 13 (7.0) | 15 (7.6) |
| moniliasis | 9 (4.8) | 14 (7.5) | 12 (6.1) |
| constipation | 9 (4.8) | 12 (6.4) | 12 (6.1) |
| fever | 10 (5.3) | 14 (7.5) | 9 (4.5) |
| dermatitis | 4 (2.1) | 17 (9.1) | 10 (5.1) |
| contact | | | |
| rash | 10 (5.3) | 10 (5.3) | 10 (5.1) |
| pneumonia | 7 (3.7) | 5 (2.7) | 14 (7.1) |

*post vaccination information not available for one subject in each group

Safety Results: Number (%) of Serious Adverse Events (SAEs)
[number of SAE considered by the investigator to be related
to study medication] (Total vaccinated cohort)

| | | |
|---|---|---|
| Separate | Staggered | Combination |
| Vaccine Group | Vaccine Group | Vaccine Group |
| N = 188 | N = 187* | N = 198* |

Serious Adverse Events throughout the entire study period including
extended safety follow-up period after last vaccine dose

| | | | |
|---|---|---|---|
| Subjects with any | 9 (4.8) [0] | 6 (3.2) [1] | 9 (4.5) [0] |
| SAE(s), n (%) | | | |
| [n related] | | | |
| asthma | 1 (0.5) [0] | 0 (0.0) [0] | 1 (0.5) [0] |
| bronchospasm | 2 (1.1) [0] | 0 (0.0) [0] | 0 (0.0) [0] |
| cellulitis | 1 (0.5) [0] | 0 (0.0) [0] | 0 (0.0) [0] |
| convulsions | 0 (0.0) [0] | 1 (0.5) [0] | 1 (0.5) [0] |
| dehydration | 1 (0.5) [0] | 1 (0.5) [0] | 0 (0.0) [0] |
| fever | 2 (1.1) [0] | 1 (0.5) [0] | 0 (0.0) [0] |
| gastroenteritis | 2 (1.1) [0] | 1 (0.5) [0] | 2 (1.0) [0] |
| gastroesophageal | 0 (0.0) [0] | 0 (0.0) [0] | 1 (0.5) [0] |
| reflux | | | |
| infection viral | 0 (0.0) [0] | 1 (0.5) [0] | 0 (0.0) [0] |
| neoplasm nos | 0 (0.0) [0] | 1 (0.5) [0] | 0 (0.0) [0] |
| neuropathy | 0 (0.0) [0] | 1 (0.5) [0] | 0 (0.0) [0] |
| otitis media | 1 (0.5) [0] | 0 (0.0) [0] | 0 (0.0) [0] |
| pneumonia | 3 (1.6) [0] | 1 (0.5) [0] | 4 (2.0) [0] |
| pneumonitis | 1 (0.5) [0] | 0 (0.0) [0] | 0 (0.0) [0] |
| pyelonephritis | 0 (0.0) [0] | 1 (0.5) [1] | 0 (0.0) [0] |
| urinary tract | 0 (0.0) [0] | 0 (0.0) [0] | 1 (0.5) [0] |
| infection | | | |

Fatal Serious Adverse Events throughout the entire study period including
extended safety follow-up period after last vaccine dose

| | | | |
|---|---|---|---|
| Subjects with fatal | 0 (0.0) [0] | 0 (0.0) [0] | 0 (0.0) [0] |
| SAE(s), n (%) | | | |
| [n related] | | | |

*post vaccination information not available for one subject in each group
Conclusion: One month after the third dose of the DTaP-HepB-IPV vaccine given concomitantly with Hib and PnC, geometric mean antibody concentrations for each of the pertussis antigens, and seroprotection rates for diphtheria, tetanus and the polioviruses, were shown to be non-inferior to those achieved following separately administered vaccines. Unsolicited adverse events were reported in 154 (81.9%) of the separate vaccine group, 164 (87.7%) of the staggered vaccine group and 165 (83.3%) of the combined vaccine group, with the most frequently reported in each group being upper respiratory tract infection and otitis media. Non-fatal serious adverse events were reported in 9 (4.8%) of the separate vaccine group, 6 (3.2%) of the staggered vaccine group and 9 (4.5) of the combined vaccine group. Pneumonia, bronchospam, fever and gastroenteritis were the only non-fatal serious adverse events reported in more than one subject in the separate vaccine group. No single non-fatal serious adverse event was reported in more than one subject in the staggered vaccine group and pneumonia and gastroenteritis were the only events reported by more than one subject in the combined vaccine group. No deaths were reported.

### REFERENCES

### The Contents of Which are Hereby Incorporated by Reference

[1] Armand et al. (1982) J. Biol. Stand. 10:335-339.
[2] Cadoz et al. (1985) Vaccine 3:340-342.
[3] MMWR (1997) 46(RR-5) 1-10.
[4] Baklaic et al. (1983) Infect. 17 nmun. 42:599-604.
[5] Jones (2001) Curr Odin Investig Drugs 2:47-49.
[6] Costantino et al. (1992) Vaccine 10:691-8.
[7] Lieberman et al. (1996) JAMS 275:1499-503.
[8] W02005/000345.
[9] W002/058737.
[10] W003/007985.
[11] Rennels et al. (2002) Pediatr Infect Dis J21:978-979,
[12] WO2004/013400.
[13] Campbell et al. (2002) JInfectDis 186:1848-1851.
[14] Herzenberg et al. (1980) Nature 285: 664-667.
[15] Schutze et al. (1985) J Immunol 135:2319-2322.
[16] Dagan et al. (1998) InfectImmun 66:2093-2098.
[17] Barington et al. (1994) InfectImmun 62:9-14.
[18] Di John et al. (1989) lances 2(8677):1415-8.
[19] Granoff et al. (1993) Vaccine Suppll: S46-51.
[20] Granoff et al. (1994) JAMA 272:1116-1121.
[21] Barington et al. (1993) Infect Immun 61:432-438.
[22] Australian patent 748716 (granted from WO98/51339).
[23] Olander et al. (2001) Vaccine 20:336-341.
[24] Burrage et al. (2002) Infect Immun 70:4946-4954.
[25] Peeters et al. (1999) InfectImmun 59:3504-3510.
[26] Hoppenbrouwers et al. (1999) Vaccine 17:2588-98.
[27] W002/00249.
[28] WO00/56360.
[29] Reddin et al. (2001) FEMSImmunol Med Microbiol 31:153-162.
[30] Podda et al. (1991) Vaccine 9:741-745.
[31] Darkes & Plosker (2002) Paediatr Drugs 4:609-630.
[32] Vaccines. (eds. Plotkin & Orenstein). 4th edition, 2004, ISBN: 0-7216-9688-0.
[33] Del Guidice et al. (1998) Molecular Aspects of Medicine 19:1-70.
[34] Anonymous (January 2002) Research Disclosure, 453077.
[35] Anderson (1983) Infect Immun 39(1):233-238.
[36] Anderson et al. (1985) J Clin Invest 76(1):52-59.
[37] Ramsay et al. (2001) Lances 357(9251):195-196.
[38] Lindberg (1999) Vaccine 17 Suppl 2:S28-36.
[39] Buttery & Moxon (2000) JR Coll Physicians Lond 34:163-168.
[40] Ahmad & Chapnick (1999) Infect Dis Clin North Am 13:113-33, vii.
[41] Goldblatt (1998) J. Med. Microbiol. 47:563-567.
[42] European patent 0477508.
[43] U.S. Pat. No. 5,306,492.
[44] WO98/42721,
[45] Dick et al. in Conjugate Vaccines (eds. Cruse et al.) Karger, Basel, 1989, 10:48-114.
[46] Hermanson Bioconjugate Techniques, Academic Press, San Diego (1996) ISBN: 0123423368.
[47] W003/080678.
[48] Glode et al. (1979) JInfect Dis 139:52-56
[49] W094/05325; U.S. Pat. No. 5,425,946.
[50] United Kingdom patent application 0323103.2.
[51] WO99/42130
[52] W096140242
[53] Lees et al. (1996) Vaccine 14:190-198.
[54] W095/08348.
[55] U.S. Pat. No. 4,882,317
[56] U.S. Pat. No. 4,695,624
[57] Porro et al. (1985) Mol Immunol 22:907-919.
[58] EP-A-0208375
[59] WO00/10599
[60] Gever et al. Med. Microbiol. Immunol, 165: 171-288 (1979).
[61] U.S. Pat. No. 4,057,685.
[62] U.S. Pat. Nos. 4,673,574; 4,761,283; 4,808,700.
[63] U.S. Pat. No. 4,459,286.
[64] U.S. Pat. No. 4,965,338
[65] U.S. Pat. No. 4,663,160
[66] U.S. Pat. No. 4,761,283
[67] U.S. Pat. No. 4,356,170
[68] Balmer & Borrow (2004) Expert Rev Vaccines 3:77-87.
[69] Watson (2000) Pediatr infect Dis J 19:331-332.
[70] Rubin (2000) Pediatr Clin North Am 47:269-285, v.
[71] Jedrzejas (2001) Alicrobiol Mol Biol Rev 65:187-207.
[72] Zielen et al. (2000) infect. Immun. 68:1435-1440.
[73] Kanra et al. (1999) The Turkish Journal of Paediatrics 42:421-427.
[74] Ravenscroft et al. (2000) Dev Biol (Basel) 103: 35-47.
[75] WO97/00697.
[76] WO96/37222; U.S. Pat. No. 6,333,036.
[77] W02004/032958
[78] Gennaro (2000) Remington: The Science and Practice of Pharmacy. 20th ed. ISBN: 0683306472.
[79] Almeida & Alpar (1996) J. Drug Targeting 3:455-467.
[80] Agarwal & Mishra (1999) Indian JExp Biol 37:6-16.
[81] Vaccine Design (1995) eds. Powell & Newman. ISBN: 030644867X. Plenum.
[82] WO00/23105.
[83] WO90/14837.
[84] U.S. Pat. No. 5,057,540.
[85] WO96/33739.
[86] EP-A-0109942.
[87] WO96/11711.
[88] WO00/07621.
[89] Barr et al. (1998) Idvanced Drug Delivey Reviews 32:247-271.
[90] Sjolanderet et al. (1998) Advanced Drug Delivery Reviews 32:321-338.
[91] Nilkura et al. (2002) Virology 293:273-280.
[92] Lenz et al. (2001) J Immunol 166:5346-5355.
[93] Pinto et al. (2003) JInfect Dis 188:327-338.
[94] Gerber et al. (2001) Virol 75:4752-4760.
[95] WO03/024480
[96] WO03/024481
[97] Glucketal. (2002) Vaccine-20:B10-B16.
[98] EP-A-0689454.
[99] Johnson et al. (1999) BioorgMed Chem Lett 9:2273-2278.
[100] Evans et al. (2003) Expert Rev vaccines 2:219-229.
[101] Meraldi et al. (2003) Vaccine 21:2485-2491.
[102] Pajak et al. (2003) Vaccine 21:836-842.
[103] Kandimalla et al. (2003) Nucleic Acids Research 31:2393-2400.
[104] W002126757.
[105] WO99/62923.
[106] Krieg (2003) Nature Medicine 9:831-835.
[107] McCluskie et al. (2002) FEMS Immunology and Medical Microbiology 32:179-185.
[108] WO98/40100.
[109] U.S. Pat. No. 6,207,646.
[110] U.S. Pat. No. 6,239,116.
[111] U.S. Pat. No. 6,429,199.
[112] Kandimalla et al. (2003) Biochemical Society Transactions 31 (part 3):654-658.
[113] Blackwell et al. (2003) J Immunol 170:40614068.
[114] Krieg (2002) Trends Immunol 23:64-65.
[115] WO01/95935.
[116] Kandimalla et al. (2003) BBRC 306:948-953.
[117] Bhagat et al. (2003) BBRC 300:853-861.
[118] W003/035836.
[119] WO95/17211.
[120] WO98/42375.
[121] Beignon et al. (2002) InfectImmun 70:3012-3019.
[122] Pizza et al. (2001) Vaccine 19:2534-2541.
[123] Pizza et al. (2000) Int J Med Microbiol 290:455-461.
[124] Scharton-Kersten et al. (2000) Infect Immun 68:5306-5313.
[125] Ryan et al. (1999) InfectImmun 67:6270-6280.
[126] Partidos et al. (1999) Immunol Lett 67:209-216.
[127] Peppoloni et al. (2003) Expert Rev Vaccines 2:285-293.
[128] Pine et al. (2002) J Control Release 85:263-270.
[129] Domenighini et al. (1995) Mol Microbiol 15:1165-1167.
[130] WO99/40936.
[131] WO99/44636.
[132] Singh et al (2001) J Cont Release 70:267-276.
[133] WO99/27960.
[134] U.S. Pat. No. 6,090,406
[135] U.S. Pat. No. 5,916,588
[136] EP-A-0626169.
[137] WO99/52549.
[138] WO01/21207
[139] WO01/21152.
[140] Andrianov et al. (1998) Biomaterials 19:109-115.
[141] Payne et al. (1998) AdvDrugDeliveryReview 31:185-196.
[142] Stanley (2002) Clin Exp Dermatol 27:571-577.
[143] Jones (2003) Curr Opin Investig Drugs 4:214-218.
[144] W004/60308
[145] WO04/64759.
[146] WO99/11241.
[147] W094/00153.
[148] W098/57659.
[149] European patent applications 0835318, 0735898 and 0761231.
[150] WO03/009869.
[151] WO2004/110480.
[152] Olander et al. (2002) Vaccine 20:336-41.
[153] McVernon et al. (2003) Vaccine 21:2573-9.
[154] Chippaux et al. (2004) Vaccine 22:3303-11.

1. A method for immunizing a human patient against a disease caused by Neisseria meningitidis, comprising the step of administering to the human patient a composition that comprises at least two of: (a) a conjugate of (i) the capsular saccharide of serogroup A N.meningitidis and (ii) a tetanus toxoid or derivative thereof; (b) a conjugate of (i) the capsular saccharide of serogroup C N.meningitidis and (ii) a tetanus toxoid or derivative thereof; (c) a conjugate of (i) the capsular saccharide of serogroup W135 N.meningitidis and (ii) a tetanus toxoid or derivative thereof; and (d) a conjugate of (i) the capsular saccharide of serogroup Y N.meningitidis and (ii) a tetanus toxoid or derivative thereof, wherein the patient has been pre-immunised with (a) a tetanus toxoid or derivative thereof and/or (b) a conjugate of (i) a capsular saccharide of an organism other than N.meningitidis and (ii) a tetanus toxoid or derivative thereof.

2. A method for immunizing a human patient against a disease caused by Neisseria meningitidis, comprising the step of administering to the human patient a composition that comprises at least two of: (a) a conjugate of (i) the capsular saccharide of serogroup A N.meningitidis and (ii) a tetanus toxoid; (b) a conjugate of (i) the capsular saccharide of serogroup C N.meningitidis and (ii) a tetanus toxoid; (c) a conjugate of (i) the capsular saccharide of serogroup W135 N.meningitidis and (ii) a tetanus toxoid; and (d) a conjugate of (i) the capsular saccharide of serogroup Y N.meningitidis and (ii) a tetanus toxoid, wherein the patient has been pre-immunised with (a) a tetanus toxoid and/or (b) a conjugate of (i) a capsular saccharide of an organism other than N. meningitidis and (ii) a tetanus toxoid.

3-4. (canceled)

5. The method of claim 1, wherein the composition comprises (a) and (b), (b) and (d), or all four of (a), (b), (c) and (d).

6. (canceled)

7. The method claim 1, wherein the patient has been pre-immunised with a vaccine comprising a tetanus toxoid.

8. The method claim 1, wherein the patient has been pre-immunised with a vaccine comprising a Hib conjugate.

9. The method claim 1, wherein the patient has been pre-immunised with a vaccine comprising at least one pneumococcal conjugate.

10. The method claim 1 wherein the patient was pre-immunised at least 0.5 month before the method.

11. The method of claim 10, wherein the patient was pre-immunised at least 8 years before the method.

12. The method claim 1, wherein the pre-immunisation took place within 1 year of the patient's birth.

13. The method claim 1 wherein the saccharides in the meningococcal conjugates (a) to (d) are shorter than the native capsular saccharides seen in meningococcus.

14. The method claim 1 wherein the meningococcal conjugates comprise a tetanus toxoid carrier and an adipic acid linker.

15. The method of claim 14, comprising no more than 50 μg of tetanus toxoid carrier.

16. The method claim 1 wherein the composition further comprises a conjugated capsular saccharide from Streptococcus pneumoniae.

17. The method claim 1, wherein the composition further comprises a conjugated capsular saccharide from Haemophilus influenzae type B.

18. The method claim 1, wherein the composition further comprises a protein antigen from serogroup B of Neisseria meningitidis.

19. The method claim 1 wherein the composition includes an aluminium hydroxide adjuvant and/or an aluminium phosphate adjuvant.

20. The method claim 1 wherein the disease caused by Neisseria meningitidis is meningococcal meningitis.

21. The method claim 1, wherein:—the patient has been pre-immunised, at least five years before the method, with a vaccine comprising a tetanus toxoid;—the meningococcal conjugates comprise a tetanus toxoid carrier and, optionally, an adipic acid linker;—the meningococcal conjugates are present at 2-15 μg/ml (measured as meningococcal saccharide) per serogroup;—the saccharide: carrier weight ratio for at least one conjugate is about 1:3; and—the composition includes 20-50 μg/ml of tetanus toxoid.

22. A method for immunizing a human patient against a disease caused by Streptococcus pneumoniae, comprising the step of administering to the human patient a composition that comprises at least seven conjugates of different capsular saccharide serotypes of pneumococcus, at least one of which is conjugated to a diphtheria toxoid or CRM197 or a derivative thereof, wherein the patient has been pre-immunised with (a) a diphtheria toxoid or derivative thereof and/or (b) a conjugate of (i) a capsular saccharide of an organism other than pneumococcus and (ii) a diphtheria toxoid or CRM197 or derivative thereof.

23. A method for immunizing a human patient against a disease caused by Streptococcus pneumoniae, comprising the step of administering to the human patient a composition that comprises at least seven conjugates of different capsular saccharide serotypes of pneumococcus, at least one of which is conjugated to tetanus toxoid or a derivative thereof, wherein the patient has been pre-immunised with (a) a tetanus toxoid or derivative thereof and/or (b) a conjugate of (i) a capsular saccharide of an organism other than pneumococcus and (ii) a tetanus toxoid or derivative thereof.

24. A method for immunizing a human patient against a disease caused by Streptococcus pneumoniae, comprising the step of administering to the human patient a composition that comprises at least seven conjugates of different capsular saccharide serotypes of pneumococcus, at least one of which is conjugated to tetanus toxoid or a derivative thereof and at least one of which is conjugated to diphtheria toxoid or CRM197 or a derivative thereof, wherein the patient has been pre-immunised with (a) a tetanus toxoid or derivative thereof and/or (b) a conjugate of (i) a capsular saccharide of an organism other than pneumococcus and (ii) a tetanus toxoid or derivative thereof and (c) a diphtheria toxoid or derivative thereof and/or (d) a conjugate of (i) a capsular saccharide of an organism other than pneumococcus and (ii) a diphtheria toxoid or CRM197 or derivative thereof.

25-27. (canceled)

28. The method claim 22, wherein a meningococcal capsular saccharide conjugate is not present in the composition.

29. The method of claim 22, wherein the patient has been pre-immunised with a vaccine comprising a diphtheria toxoid.

30. The method claim 22, wherein the patient has been pre-immunised with a vaccine comprising a tetanus toxoid.

31. The method of claim 22, wherein the patient has been pre-immunised with a vaccine comprising a Hib conjugate.

32. The method of claim 22, wherein the patient has been pre-immunised with a vaccine comprising at least one meningococcal capsular saccharide conjugate.

33. The method claim 22, wherein the patient was pre-immunised at least 0.5 month before the method.

34. The method of claim 33, wherein the patient was pre-immunised at least 8 years before the method.

35. The method claim 22, wherein the pre-immunisation took place within 1 year of the patient's birth.

36. The method of claim 22, wherein at least one of the saccharides in the pneumococcal conjugates is shorter than the native capsular saccharides seen in pneumococcus.

37. The method of claim 22, wherein at least one of the pneumococcal conjugates comprises a diphtheria toxoid carrier and, optionally, an adipic acid linker.

38. The method claim 22, wherein at least one of the pneumococcal conjugates comprises a CRM197 carrier and, optionally, an adipic acid linker.

39. The method of claim 23, wherein at least one of the pneumococcal conjugates comprises a tetanus toxoid carrier and, optionally, an adipic acid linker.

40. The methods of claim 37 or 38 claim 37 comprising no more than 60 μg of diphtheria toxoid or CRM197 carrier.

41. The method of claim 39, comprising no more than 50 μg of tetanus toxoid carrier.

42. The method of claim 22, wherein the composition further comprises a conjugated capsular saccharide from Haemophilus influenzae type B.

43. The method claim 22 wherein the composition further comprises a protein antigen from serogroup B of Neisseria meningitidis.

44. The method of claim 22, wherein the composition includes an aluminium hydroxide adjuvant and/or an aluminium phosphate adjuvant.

45. A method for immunizing a human patient against a disease caused by Neisseria meningitidis, Bordetella pertussis, Clostridium tetani, Corynebacterium diphtheriae and Streptococcus pneumoniae comprising the step of administering to the human patient the following vaccines with the following administration scheme:

| | Visit 1 | Visit 2 | Visit 3 | Visit 4 |
|---|---|---|---|---|
| DTP | X | | X | |
| Strep | | X | | X |
| MenC | | X | | X |

wherein the visit to the medical practitioner all occur in the first 8 months of life,
wherein there is at least 2 weeks between each consecutive visit,
wherein DTP comprises DT, TT, and either whole cell (Pw) or acellular (Pa) pertussis antigens,
wherein Strep is a multivalent pneumococcal capsular saccharide conjugate vaccine comprising at least 7, 10, 11, 13 or 14 conjugated serotypes,
wherein MenC comprises a conjugated N. meningitidis serogroup C capsular saccharide,
wherein at least one conjugated saccharide in each of the Strep and MenC vaccines is conjugated to DT or CRM197, or at least one conjugated saccharide in each of the Strep and MenC vaccines is conjugated to TT.

46. A method for immunizing a human patient against a disease caused by Neisseria meningitidis, Bordetella pertussis, Clostridium tetani, Corynebacterium diphtheriae and Streptococcus pneumoniae comprising the step of administering to the human patient the following vaccines with the following administration scheme:

| | Visit 1 | Visit 2 | Visit 3 | Visit 4 |
|---|---|---|---|---|
| DTP | X | X | X | |
| Strep | X | | X | |
| MenC | | X | | Optionally X |

wherein the visit to the medical practitioner all occur in the first 8 months of life,
wherein there is at least 2 weeks between each consecutive visit,
wherein DTP comprises DT, TT, and either whole cell (Pw) or acellular (Pa) pertussis antigens,
wherein Strep is a multivalent pneumococcal capsular saccharide conjugate vaccine comprising at least 7, 10, 11, 13 or 14 conjugated serotypes,
wherein MenC comprises a conjugated N. meningitidis serogroup C capsular saccharide,
wherein at least one conjugated saccharide in each of the Strep and MenC vaccines is conjugated to DT or CRM197, or at least one conjugated saccharide in each of the Strep and MenC vaccines is conjugated to TT.

47. A method for immunizing a human patient against a disease caused by Neisseria meningitidis, Bordetella pertussis, Clostridium tetani, Corynebacterium diphtheriae and Streptococcus pneumoniae comprising the step of administering to the human patient the following vaccines with the following administration scheme:

| | Visit 1 | Visit 2 | Visit 3 |
|---|---|---|---|
| DTP | X | X | X |
| Strep | X | X | |
| MenC | | X | X |

wherein the visit to the medical practitioner all occur in the first 8 months of life,
wherein there is at least 2 weeks between each consecutive visit,
wherein DTP comprises DT, TT, and either whole cell (Pw) or acellular (Pa) pertussis antigens,
wherein Strep is a multivalent pneumococcal capsular saccharide conjugate vaccine comprising at least 7, 10, 11, 13 or 14 conjugated serotypes,
wherein MenC comprises a conjugated N. meningitidis serogroup C capsular saccharide,
wherein at least one conjugated saccharide in each of the Strep and MenC vaccines is conjugated to DT or CRM197, or at least one conjugated saccharide in each of the Strep and MenC vaccines is conjugated to TT.

48. The method of claim 45, wherein Visits 1, 2 and 3 occur at: 2, 3, 4 months of age; 3, 4, 5 months of age; 2, 4, 6 months of age; or 6, 10, 14 weeks of age.

49. The method of claim 45, wherein visit 4 occurs at 5 months of age.

50. The method of claim 45, wherein DTP, MenC and Strep are administered as separate injections at any one visit.

51. The method of claim 45, wherein DTP, MenC and Strep are administered as a combination vaccine at any one visit.

52. The method of claim 45, wherein MenB, a N. meningitidis subunit protein vaccine or a N. meningitidis outer membrane vesicle vaccine is administered at Visit 1 and Visit 3.

53. The method of claim 52, wherein MenB is given as a booster dose at a further visit at 11-15 months of age.

54. The method of claim 45, wherein DTP is given as a booster dose at a further visit at 11-15 months of age.

55. The method of claim 45, wherein MenC is given as a booster dose at a further visit at 11-15 months of age.

56. The method of claim 45 wherein Strep is given as a booster dose at a further visit at 11-15 months of age.

57. The method of claim 45 wherein DTP further comprises HepB surface antigen.

58. The method of claim 45, wherein DTP further comprises inactivated polio virus.

59. The method of claim 45, wherein MenC further comprises a conjugated N. meningitidis serogroup A capsular saccharide.

60. The method of claim 45 wherein MenC further comprises a conjugated N. meningitidis serogroup Y capsular saccharide.

61. The method of claim 45 wherein MenC further comprises a conjugated N. meningitidis serogroup W135 capsular saccharide.

62. The method of claim 45 wherein MenC further comprises a conjugated H. influenzae type B capsular saccharide.

63. The method of claim 45 wherein DTP further comprises a conjugated H. influenzae type B capsular saccharide.

64. The method of claim 45, wherein at least one capsular saccharide is conjugated to TT in the MenC and Strep vaccines.

65. The method of claim 45, wherein at least one capsular saccharide is conjugated to DT in the MenC and Strep vaccines.

66. The method of claim 45 wherein at least one capsular saccharide is conjugated to CRM197 in the MenC and Strep vaccines.

67. (canceled)

68. A kit comprising all the vaccines required for Visit 1 vaccine administration of claim 45 and instructions for its use.

69. A kit comprising all the vaccines required for Visit 2 vaccine administration of claim 45 and instructions for its use.

70. A kit comprising all the vaccines required for Visit 3 vaccine administration of claim 45 and instructions for its use.
